(19) United States
(12) Patent Application Publication
(10) Pub. No.: US 2003/0064507 A1
(43) Pub. Date: Apr. 3, 2003

(54) System and methods for mixing within a microfluidic device

(76) Inventors: Sean Gallagher, Claremont, CA (US); Roberta Druyor-Sanchez, Mesa, AZ (US); Yuk-Tong Chan, Scottsdale, AZ (US); David Dorris, Austin, TX (US); Lawrence Dues, Chandler, AZ (US); Alan Paul De La Cerda, Chandler, AZ (US); Norb Simonson, Mesa, AZ (US); Clifford Lynde Hunt Anderson, Tempe, AZ (US); Phillip Franciskovich, Phoenix, AZ (US); Peter Albert Kahn, Phoenix, AZ (US)

Correspondence Address: Robin M. Silva, Esq., DORSEY & WHITNEY, LLP, Suite 3400, Four Embarcadero Center, San Francisco, CA, 94111-4187, US

(21) Appl. No.: 10/206,841

(22) Filed: Jul. 26, 2002

Related U.S. Application Data
(60) Provisional application No. 60/395,257, filed on Jul. 11, 2002.
(60) Provisional application No. 60/308,169, filed on Jul. 26, 2001.

(51) Int. Cl. C12M 1/34

(52) U.S. Cl. 435/287.2; 435/6; 422/99

(57) ABSTRACT

The present invention provides microfluidic systems comprising microfluidic chambers and mixers, and methods of use. The microfluidic chambers of the present invention comprise a flexible membrane which provides efficient mixing of the solution therein. The present invention also provides a microfluidic chamber in fluidic communication with a micro-disk and a microfluidic chamber comprising a shim such that and a contiguous gap is present between a sample fluid and the chamber membrane. The microfluidic systems find use in the decrease in time for reactions occurring therein.

This application claims the benefit of the filing dates of U.S. patent application Ser. No. 60/395,257, filed Jul. 11, 2002 and U.S. patent application Ser. No. 60/308,169, filed Jul. 26, 2001, both applications are expressly incorporated by reference.

### FIELD OF THE INVENTION

The present invention is directed to novel microfluidic systems and methods of use to enhance the mixing of solutions within a microfluidic chamber.

### BACKGROUND OF THE INVENTION

Advances in molecular biology have provided methods of identifying pathogens, diagnosing disease states, and performing forensic determinations using gene sequences and polypeptides. A concomitant need has arisen for equipment that performs these methods in a high-capacity, miniaturized, and automated format. Microfluidic chambers have been developed for these purposes.

Most, if not all, reactions performed in microfluidic chambers require mixing of the reaction components. For example amplification of nucleic acid by the polymerase-chain-reaction (PCR) requires mixing DNA template, primers, buffer, polymerase, nucleotides etc. needed for DNA synthesis. Mixing also is required for efficient hybridization of a target nucleic acid to a probe array attached to a surface within a microfluidic chamber. Simply adding the reaction components separately to a microfluidic chamber generally does not result in effective mixing, as microfluidic flow is substantially laminar. Therefore, without mixing, the reaction rates are generally limited by the rate of diffusion. An additional impediment to achieving efficient reaction rates are the minute quantities (e.g. <picomole) of a target analyte obtained in biological samples. Therefore, in the absence of efficient mixing of the reaction components, tens of hours may be required for a detectable result to be obtained.

In U.S. Pat. No. 6,050,719, Winkler et al. attempted to address reagent mixing limitations within a microfluidic chamber. The chamber described by Winkler et al. is defined by two plates narrowly spaced apart and manufactured from rigid materials, glass or silicon. The reaction solution entirely filled the chamber. Winkler et al. placed the chamber in a rotating box with the axis of rotation being perpendicular to the face of the plates. Winkler et al. suggested that rotation of the chamber will cause the fluid to become agitated as the direction of flow is hindered due to the change in direction of the walls of the chamber. However, Winkler et al. failed to describe that the fluid within the chamber only moves very slightly due to the high surface tension between the fluid and the chamber surfaces in the absence of a bubble in the chamber.

In U.S. Pat. No. 6,170,981, Regnier et al. described micromachined obstacles in a channel that are designed to create vortices and ideally turbulent flow thereby causing the fluids within the channel to mix. This method has two disadvantages. First, it requires additional manufacturing steps increasing the overall cost and complexity of the device. Second, it will not work in a bulk microfluidic reaction chamber, such as that required for hybridization reactions to an oligonucleotide array.

In U.S. Pat. No. 6,114,122, Besemer et al. describe a number of different mechanisms for mixing a hybridization solution within a microfluidic device such as PZT ultrasonic mixing, and pumping a hybridization solution in and out of a microfluidic chamber. Bresemer et al. also describe placing a gas bubble in a microfluidic chamber containing a hybridization solution, and agitating the device. The movement of the gas bubble in the chamber causes mixing. An obvious drawback is that the gas bubble can interfere with the even distribution of the sample over an array of capture probes, resulting in an unacceptable decrease in reaction reproducibility and efficiency.

Therefore, reasons exit for the avoidance of bubble formation within a microfluidic chamber. Bubble formation has a number of causes, one of which can be the introduction of a sample into a microfluidic chamber. For example, bubbles may form when a flexible membrane of a microfluidic chamber touches and adheres to a substrate that defines the bottom of the chamber, and on which an array of capture probes is located. Adding the liquid sample to the chamber usually causes the flexible membrane to lift unevenly resulting in air being trapped within chamber. Dividing the chamber into several smaller chambers alleviates the problem because the flexible membrane does not sag sufficiently to touch the substrate. However, in many cases it is not desirable to divide the chamber.

Thus, there remains a need in the art for devices and methods for more efficient mixing of reaction solutions within a microfluidic chamber, while at the same time maintaining the consistency and reliability of the reaction, and keeping the device construction relatively simple. There is also a need for a simple device and method for reducing the amount of bubble formation in a flexible membrane microfluidic reaction chamber, other than making the reaction chamber smaller.

### SUMMARY OF THE INVENTION

In accordance with these objectives, the present invention provides a microfluidic system comprising a microfluidic chamber comprising a flexible membrane adhered to a first surface of a substrate, a first port, and a mixer.

In another embodiment, the present invention provides a microfluidic system comprising a microfluidic chamber enclosing an area of a first surface of a substrate and a micro-disk in fluidic communication with the chamber.

In another embodiment, the present invention provides a microfluidic system comprising a microfluidic chamber comprising a membrane, a spacer, a substrate and a mixer, wherein a contiguous gap is maintained between the upper inner surface of the membrane and a sample fluid within the chamber.

In another embodiment, the present invention provides a microfluidic system comprising a mixer and first and second microfluidic chambers comprising a flexible membrane and first and second substrates, wherein opposite sides of the membrane are adhered to and enclose areas on both substrates such that both areas are in fluidic communication, and wherein one of the chambers comprises a first port.

In another embodiment, the present invention provides a microfluidic system comprising first and second microfluidic chambers comprising a membrane, and first and second substrates, wherein opposite sides of the membrane are adhered to the first and second substrates and enclose first and second areas of said substrates, wherein the first and second areas are in fluidic communication, and one of the chambers comprises a first port; and a micro-disk in fluidic communication with at least one chamber.

In another embodiment, the present invention provides a microfluidic system comprising first and second microfluidic chambers comprising a flexible membrane, and a substrate, wherein the membrane is adhered to the substrate and encloses first and second areas of the substrate, wherein the first and second areas are in fluidic communication, and one of the chambers comprises a first port; and a mixer.

In another embodiment, the present invention provides microfluidic system comprising first and second microfluidic chambers comprising a membrane, and a substrate, wherein the membrane is adhered to the substrate and encloses first and second areas of the substrate, wherein the first and second areas are in fluidic communication, and one of the chambers comprises a first port; and a micro-disk in fluidic communication with at least one chamber.

In another aspect, the present invention provides a method of mixing a fluid in a microfluidic chamber comprising a flexible membrane by applying a force to the flexible membrane.

In another aspect, the present invention provides a method of mixing a fluid in a microfluidic chamber by applying a force to the fluid using a micro-disk in fluidic communication with the chamber.

In further aspects, the present invention provides a flexible membrane comprising a dome or polypropylene, or supported by a support structure; a micro-disk regulated by a magnetic field generator, wherein the generator is either “on-” of “off chip”; mixer of various types, positioned to a apply a force either director or indirectly to a flexible membrane, wherein the force is preferably variable and is selected from the group consisting of centrifugal, lateral and rotational; various types of mixers; substrates comprised of ceramic, glass, or silicon and optionally comprising an array of capture probes; ports and other devices that provide microfluidic communication; a microfluidic chamber having an inner surface comprising hydrophilic and hydrophobic regions; a microfluidic chamber comprising low surface energy plastics; and a surfactant.

### DESCRIPTION OF THE DRAWINGS

FIG. 1 depicts one embodiment of microfluidic system 10 having rotary shaker 20, substrate 30, adhesive layer 40, flexible membrane 50 (cantilevered, not shown), adhesive layer 60, and top layer 70. Substrate 30 has an array 31 of biological binding molecules 32 attached thereto. Adhesive layer 40 has void 43, first notch 41 and second notch 42 removed therefrom. Flexible membrane 50 has first hole 51 and second hole 52 removed therefrom. Flexible membrane 50, adhesive layer 40, and substrate 30 together define microfluidic chamber80. Adhesive layer 60 has void 63, first hole 61, and a second hole 62 removed therefrom. Top layer 70 has void 73, first notch 71, and second notch 72 removed therefrom. First port 90 of microfluidic chamber 80 is defined by the alignment of first notch 41, first hole 51, first hole 61, and first notch 71. Second port 100 of microfluidic chamber 80 is defined by the alignment of second notch 42 second hole 52, second hole 62, and second notch 72.

FIG. 2 depicts a cross section of microfluidic system 10 showing rotary shaker 20, substrate 30, biological binding molecules 32, adhesive layer 40, flexible membrane 50, adhesive layer 60, and top layer 70.

FIG. 3 depicts one embodiment of microfluidic system 110having substrate 120, adhesive layer 130, micro-disk 140, and rigid membrane 150. Adhesive layer 130 has first void 131, flow channel 132, and second void 133, removed therefrom. Microfluidic chamber 160 is defined by substrate 120, adhesive layer 130, and rigid membrane 150. Membrane 150 has first hole 151 and second hole 152 removed therefrom, which function as ports.

FIG. 4 depicts one embodiment of microfluidic system 170 having substrate 180, adhesive layer 190, micro-disk 200, and rigid membrane 210. Adhesive layer 190 has first void 191 and second void 192, removed therefrom. Substrate 180, adhesive layer 190, and rigid membrane 210 define microfluidic chamber 220. Rigid membrane 210 has hole 211 removed therefrom, which functions as a port. Flow channel 212 is on the top surface of rigid membrane 210. Micro-disk 200 is caged or housed on the underside of rigid membrane 210

FIG. 5 depicts one embodiment of microfluidic system 230 having substrate 240, substrate 250, adhesive layer 260, adhesive layer 270, micro-disk 280, and rigid membrane 290. Adhesive layer 260 has first void 261 and second void 262 removed therefrom. Adhesive layer 270 has first void 271 and second void 272 removed therefrom. Microfluidic chamber 300 is defined by substrate 240, adhesive layer 260, and rigid membrane 290. Microfluidic chamber 310 is defined by substrate 250, adhesive layer 270, and rigid membrane 290. Rigid membrane 290 has void 291 with micro-disk 280 therein and slit 292 and slit 293 which function as ports. Micro-disk 280 is connected to channel 294, channel 295 and slit 293. Reagent is pumped between chamber 300 and chamber 310 through channel 294, channel 295, and slit 293. Slit 292 and slit 293 are covered by tape (not shown) during mixing. Thus, microfluidic chambers 310 and 310, are in fluidic communication.

FIG. 6 is a graph of percent area mixed in two experiments using microfluidic chambers having a total volume of 250 μl and comprising a flexible membrane versus time without applying a force to the chamber.

FIG. 7 is a graph of percent area mixed in microfluidic chambers having the indicated volumes and microfluidic chambers having domed membranes versus time with a force applied to the chamber.

FIG. 8A is a graph of fluorescent signal detected at the indicated positions in a probe array within a microfluidic chamber comprising a flexible membrane. A Cy3 labelled target nucleic acid was incubated in a microfluidic chamber comprising a capture probe array for 18 hour-hybridization without shaking.

FIG. 8B is a graph of fluorescent signal detected at the indicated positions in a probe array within a microfluidic chamber comprising a flexible membrane. A Cy3 labelled target nucleic acid was incubated in a microfluidic chamber comprising a capture probe array for 18 hour-hybridization with shaking.

FIG. 9 is a graph of percent spread area versus time of a target nucleic acid in a microfluidic chamber comprising a microarray under the indicated conditions.

FIG. 10 depicts the reactants for linear polymer synthesis by a free radical initiator: acrylamide (I) (main backbone polymer); acrylamide with NHS ester oligonucleotide attachment site (II); acrylamide with benzophenone crosslinking agent (III). The percentage of the reactants 89-94% (I), 5-10% (II); and <1% (III).

FIG. 11 depicts the structure of a linear polymer product produced by the reaction depicted in FIG. 10. The benzophenone of (III) and the NHS of (II) are boxed.

FIG. 12 depicts the attachment of a linear polymer to a substrate surface (silanized glass) using UV light as an initiator.

FIG. 13 depicts the crosslinked linear polymer attached to a substrate.

FIG. 14 depicts the oligonucleotide coupling reaction to a linear polymer.

FIG. 15 depicts one embodiment of microfluidic system 351 having microfluidic chamber 350 defined by flat sheet membrane 380, perimeter shim 410 having void 431 therein, to which perimeter adhesives 410 and 420, are attached, and substrate 440 having an array 430. Flat sheet membrane 380 has ports 360 and 390 at opposite ends. Ports 360 and 390 are sealed by tape 370 after sample fluid loading.

FIG. 16 depicts one embodiment of microfluidic system 541 having microfluidic chamber 540 defined by flat sheet membrane 550, perimeter shim 570, to which perimeter adhesives 560 and 580, are attached, and substrate 590 having arrays 581, 582, 583, and 584. Perimeter shim 570 has voids 521, 522, 523, and 524 therein. Flat sheet membrane 550 has ports 520-523 and 530-533 at opposite ends.

FIG. 17 depicts one embodiment of microfluidic system 451 having microfluidic chamber 501 defined by perimeter shim 450, adhesive layer 480 having void 490, and substrate 510 having array 500. Perimeter shim 450 is contiguous with the membrane 481, which has ports 460 and 470 at opposite ends. Ports 460 and 470 are sealed by tape 452 after target loading.

FIG. 18 depicts one embodiment of microfluidic system 651 having microfluidic chamber 601 defined by perimeter shim 620, adhesive layer 640 having voids 630, 631, 632, and 633 therein, and substrate 650 having arrays 660, 661, 662, and 663. Perimeter shim 620 is contiguous with membrane 621, which has ports 600-603 and 610-613.

### DETAILED DESCRIPTION OF THE PREFERRED EMBODIMENTS

The present invention is directed to microfluidic systems and methods of use. The microfluidic systems comprise microfluidic chambers with improved mixing of solutions within the chamber and therefore improved processing and detection of target analytes.

In one embodiment, the invention provides a microfluidic system comprising a microfluidic chamber at least comprising a flexible membrane and a mixer. For example, the mixer may be a rotary type shaker. As a result of the flexibility of the membrane, mixing results in the deformation of the membrane in different directions over time, allowing the fluid within the chamber to actually mix.

In another embodiment, the invention provides a microfluidic system comprising a micro-disk in fluidic communication with a microfluidic chamber. In this embodiment, the chamber comprises a micro-disk that rotates upon the introduction of energy, such as a magnetic stir bar and a magnetic, for example an electromagnet. That is, by applying energy to allow the micro-disk or micro-bar to rotate, move or vibrate, mixing of the fluid within the chamber is accomplished. As is more fully described herein, the chamber may be divided, to allow the micro-disk to be confined within a particular area of the chamber, for example away from an array of capture probes, to prevent damage to the array. Alternatively, the micro-disk may be within the main body of the chamber.

In another embodiment, the invention provides a microfluidic system comprising a microfluidic chamber and a mixer. The microfluidic chamber comprises a substrate, a membrane, and a spacer, such that a continuous gap exists between a sample fluid within the chamber and the member. Mixing is achieved by the application of a force from a mixer. The air gap permits fluid displacement and mixing by the applied force.

In other embodiments, the invention provides combinations of these systems. In yet other embodiments, the invention provides microfluidic systems in combination with other microfluidic devices, modules, or components. The invention further provides methods of mixing a fluid sample in a microfluidic chamber. The advantages of the present invention include improved reagent exchange throughout a microfluidic chamber thereby decreasing reaction time while increasing reaction efficiency in the detection of a target analyte.

In another aspect of the invention, weight bearing flexible membranes are provided which substantially decreases the amount of gas or air inadvertently trapped in a microfluidic chamber upon the introduction or removal of a sample fluid.

By “microfluidic system” and grammatical equivalents herein are meant a microfluidic chamber and a mixer, wherein the mixer is configured to apply a force such that the contents of a microfluidic chamber are appropriately mixed.

By “mixing” and grammatical equivalents herein are meant to circulate or agitate a fluid such that at least one substance in the fluid is distributed, preferably but not required to be evenly within an area or a volume. Accordingly, mixing includes, for example, the circulation or agitation of a fluid, causing a more even distribution of at least one substance, whether particulate, dissolved or suspended, in the fluid. Within the definition of mixing also is contemplated the continued circulation or agitation of a fluid, even though the continued mixing does not further distribute a substance within the fluid. Thus, in a preferred embodiment, mixing results in a fluid that is spatially homogeneous or uniform. The degree of mixing, the timing and the force applied to effectuate the mixing are selected at the discretion of the practitioner based on the target analyte, the sample, the detection method etc. as known in the art.

By “microfluidic chamber”, “chamber” and grammatical equivalents herein are meant a device comprising a space or volume suitable for manipulating or containing small amounts of fluid, ranging from nanoliters to milliliters, although in some applications larger or smaller fluid volume will be necessary. Preferably, a microfluidic chamber comprises a membrane adhered to a substrate, defining the chamber, and allows improved mixing of a fluid within the chamber, as further described below. The microfluidic chambers of the invention can be configured in a variety of ways, as will be appreciated by those in the art. In one embodiment, the chamber is formed from a planar or flat substrate, and an intervening layer such as an adhesive layer or a layer of spacer material (a silicone sheet, etc.), with a flexible membrane cover. As outlined herein, the flexible membrane may also be thermoformed to make a “dome” shape, further defining the microfluidic chamber. Alternatively, the substrate may have an indentation in it, covered by the flexible membrane. In addition, combinations of these three embodiments can be used. In other embodiments, the microfluidic chambers provided additionally may be used for other functions selected at the discretion of the practitioner, such as, storage of reagents or samples; the contact of a fluid within the chamber with an electrode, a physical constriction, an array of binding molecules, or a detection module, and the like as further described below. In some embodiments, the microfluidic chamber is suitable for performing chemical, biochemical, or biological reactions, including amplification reactions for the detection of a target analyte. In one embodiment, a microfluidic chamber may be a closed or self-contained device. Alternatively, a microfluidic chamber may be in fluidic communication with other chambers, devices, modules, or the exterior of the chamber as described below. Structures within a microfluidic chamber generally have dimensions on the order of microns, although in many cases larger dimensions on the order of millimeters, or smaller dimensions on the order of nanometers, are advantageous. In general, chamber sizes range from 1 nl to about 1 ml, with from about 1 μl to about 250 μl being preferred and from about 10 μl to about 100 μl being especially preferred. Generally, the microfluidic chambers of the invention and other devices that contact a sample fluid are easily sterilizable.

By “membrane” and grammatical equivalents herein are meant a component of a microfluidic chamber adhered directly or indirectly to a substrate that demarcates an area on the substrate and defines at least in part the volume of a microfluidic chamber. Thus, membranes attached to a substrate by one or more of an adhesive layer, spacer layer or a perimeter shim, as described below, are contemplated by the invention. In one embodiment, the membrane is entirely closed. In alternative embodiments, the membrane comprises channels, ports, ducts, valves, docking mechanisms, vias and the like to provide fluidic communication with other devices, chambers or modules; or to provide a means of access into the chamber, as further described below. Preferably, the membrane is gas permeable or diffusible thereby allowing the removal of gas, at the discretion of the practitioner, trapped in the chamber preferably by the application of a vacuum. Preferably the pore size is between 0.2 μm and 3.0 μm, more preferably between 0.2 μm and 1 μm, and most preferably about 0.2 μm. A membrane may be of any shape, such as, square, rectangular, triangular, circular, oval, conical, spherical, cylindrical, a dome, a sheet that is flat or irregularly shaped etc. and the like. Thus, a membrane preferably has sufficient rigidity to support its own weight, however, it is flexible enough to be deformed during mixing under certain conditions defined herein. In accordance with this embodiment, a membrane may be provided with a support structure, such as, a ridge, spine, corrugation and the like that is either internal, external or a component within the membrane. In this manner, a microfluidic chamber may be made larger without the membrane collapsing. In a preferred embodiment a membrane is molded during manufacture to comprise a support structure. The membrane also preferably is optically clear and withstands temperatures of between 50° C. and 95° C. for a period of between 8 to 12 hours without shrinkage.

By “flexible membrane” and grammatical equivalents herein are meant a membrane of a microfluidic chamber that under appropriate conditions is substantially extended or distorted without mechanically failing, resulting in the mixing of a sample fluid within the chamber. Preferably, a flexible membrane is elastic, such that upon the application of an appropriate force the shape of the membrane is temporarily distorted and upon removal of the force the membrane substantially returns to its form prior to the application of the force. The force may be applied directly or indirectly to the membrane, as further described below. Accordingly, a flexible membrane preferably comprises an elastic material, such as, nylon, plastics, such as, polypropylene, polyethylene, polyvinylidene chloride, polyester, and polystyrene, Kevlar™, Spectra®, Vectran™, elastomers (e.g. rubber, synthetic rubber, and thermoplastic elastomers) or combinations thereof. Preferably, a flexible membrane comprises polypropylene. A flexible membrane can be of any shape, as described above. In a preferred embodiment, the flexible membrane bears its own weight and therefore reduces the amount of gas or air that may be trapped in the chamber. Accordingly, in one preferred embodiment, the flexible membrane comprises a dome. In another preferred embodiment, the flexible membrane comprises a support structure.

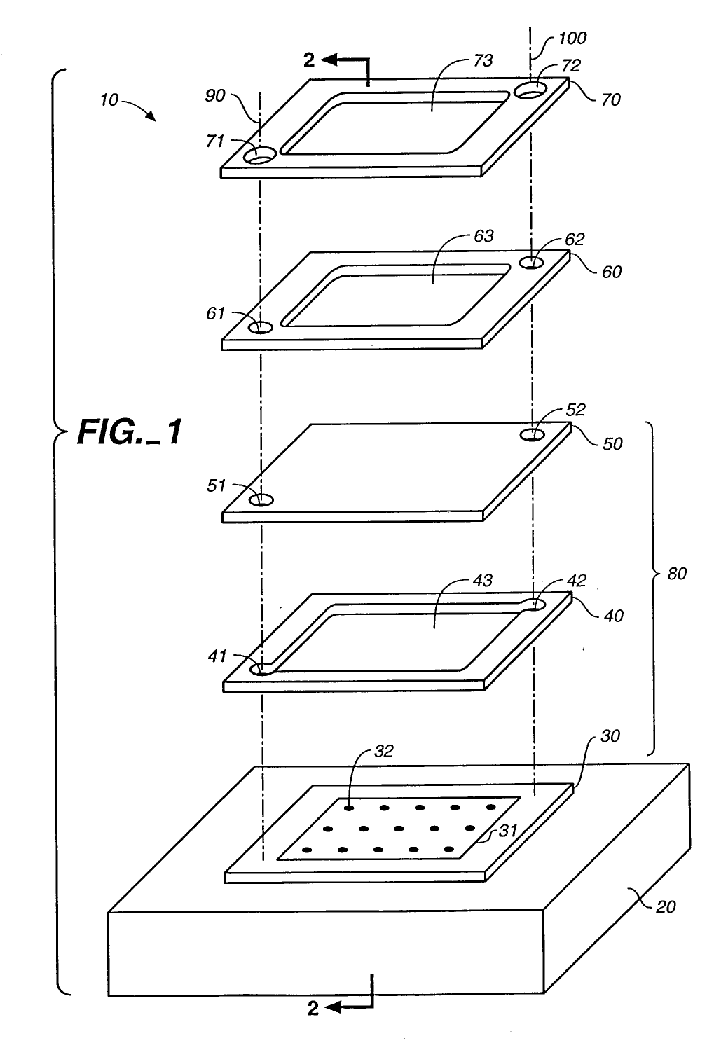

Preferably, a flexible-membrane is thermoformed to a dome shape. For example, and without limitation, a top layer, a flexible membrane, and an adhesive layer are placed against a vacuum chuck, with the top layer against the chuck. By “top layer” and grammatical equivalents herein are meant an optional component of a microfluidic chamber that finds use in the formation of a domed flexible membrane as described below. A top layer has a thickness selected at the discretion of the practitioner. A top layer may comprise any material but preferably is heat resistant such that it does not appreciably deform in the manufacture of a flexible membrane. In the embodiment depicted in FIG. 1, top layer 70 has void 73 removed therefrom and is attached to flexible membrane 50 by adhesive layer 60. Thus, a small gap exists between the vacuum chuck and flexible membrane 50 defined by the thickness of the top layer 70 and adhesive layer 60. Generally, hot gas (preferably air) is blown against the flexible membrane, and the vacuum pulls the flexible membrane into the gap. The heat allows the material to stretch inelastically under stress. In a preferred embodiment, the flexible membrane is made from a 0.004″ thick cast polypropylene material (non-oriented). The vacuum chuck preferably has a flat surface with vacuum slot dimensions of (0.004″ to 0.008″)×(0.05″ to 0.15″); although, as will be appreciated, other dimensions will be appropriate. For example, and not by way of any limitation, the vacuum chuck may have 0.004″ to 0.008″ holes, or with larger vacuum holes or slots covered by perforated sheet metal with the appropriate hole or slot size. The surface of the chuck in contact with the top layer is preferably a thermal insulator or thermal conductor held at a relatively cool temperature in order to prevent distortion of top layer. A heat shield is preferably used to limit heat transfer to the perimeter area, where the adhesive layer is otherwise exposed. The vacuum chuck surface may be designed to achieve corrugation ridges or ribs, including a logo, thereby stiffening the flexible membrane. Heat processing may also reduce electrostatic charge on the surface of the flexible membrane, reducing electrostatic force on the same, thus, reducing deflection of the flexible membrane toward the substrate. Additionally, for polypropylene the stretching may increase stiffness of the material by a process known to the artisan as “orienting”.

By “rigid membrane” and grammatical equivalents herein are meant a membrane of a microfluidic device that is inelastic and substantially maintains its shape upon the direct or indirect application of a force to the membrane. Accordingly, a rigid membrane preferably comprises inelastic material, such as, glass, or plastic and is of sufficient thickness or density to render the membrane inelastic. Examples include ABS, PVC, polyethylene, Teflon™, Kalrez™ (e.g. U.S. Pat. No. 5,945,333, incorporated by reference). Those skilled in the art are aware that an otherwise elastic material may be modified or used at a sufficient thickness or density or another manner such that it is rendered substantially inelastic.

In an optional embodiment, a microfluidic chamber further comprises a “label layer” that is cut in the same manner as the adhesive layer, described below, to form windows that correspond in location to the arrays on the substrate surface. A label layer is preferably a thick film having a layer of adhesive and is most preferably an Avery laser label. The label layer is applied to the outer surface of the membrane. The substrate surface is preferably visible through a void or window through the label layer.

By “spacer layer” and grammatical equivalents herein are meant a component of a microfluidic chamber that at least in part defines the volume of a microfluidic chamber. Accordingly, a spacer layer preferably increases the volume of a microfluidic chamber than would be achieved in the absence of the spacer layer. Thus, in one embodiment, a spacer layer, defines at least a part of the walls of a microfluidic chamber, such as a shim, and has a void therein. As shown in FIG. 16, perimeter shim 410 defines the sides or walls of the microfluidic chamber and provides a fit or connection between the membrane and the substrate. Accordingly, the spacer layer preferably comprises an adhesive as described below or is attached to the substrate and membrane by adhesive layers.

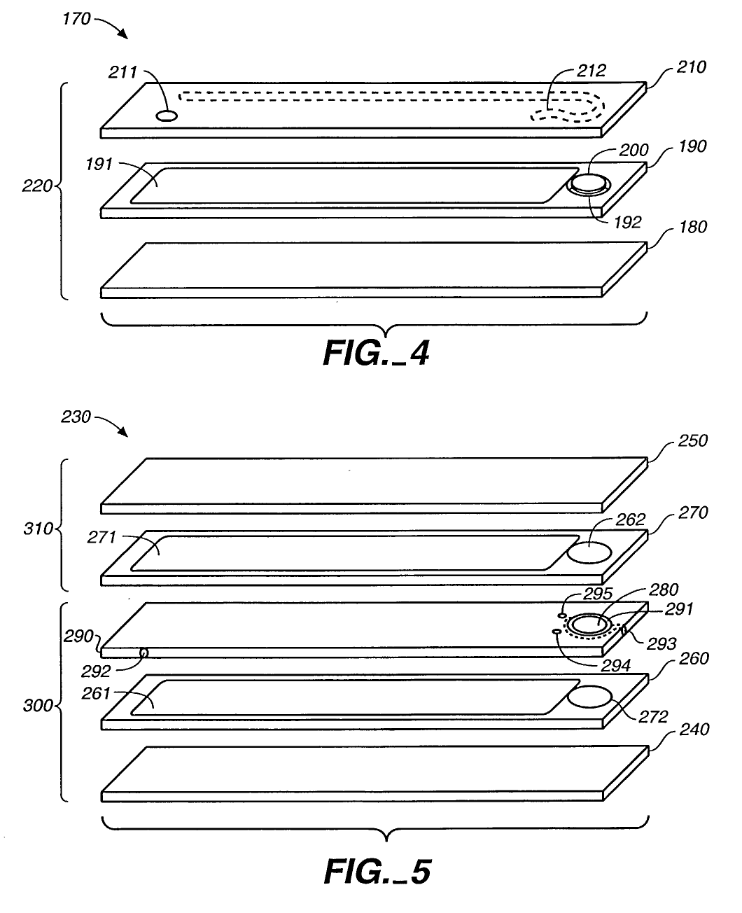

By “adhesion layer”, “adhesive layer”, and grammatical equivalents herein are meant a substance or compound that adheres a membrane and substrate of a microfluidic device together to both provide a microfluidic chamber and to a provide a seal that substantially prevents leakage of the contents of the microfluidic chamber. As will be appreciated by those in the art this may take on a variety of different forms. In one embodiment, there is a gasket, spacer layer, a shim and the like between the substrate surface and the membrane comprising sheets, tubes or strips. Alternatively, there may be a rubber or silicone strip or tube; for example, the substrate surface may comprise an indentation or channel into which the gasket fits and the membrane and substrate are clamped together. In another embodiment, adhesives are used to attach the membrane to the substrate. Examples of adhesives include a double-sided sheet, rubber adhesives, and liquid adhesives, such as silicon, acrylic, and combinations thereof. In a preferred embodiment, the adhesive layer is a sheet (e.g. 9490LE, 3M Corp.) with voids therein to further demarcate the area of the substrate within the chamber and the chamber volume, to optionally provide ports for chamber access, or flow channels for fluidic communication with other components and devices. FIG. 1 depicts one embodiment of adhesive layer 40 having void 43, first notch 41 and second notch 42 removed therefrom. Void 43 demarcates an area on substrate 30. First notch 41 and second notch42 form part of ports 90 and 100. FIG. 4 depicts adhesive layer 130 having first void 131, flow channel 132, and second void 133, removed therefrom. First void 131 demarcates an area on substrate 120. Second void 132 provides housing for micro-disk 140. Adhesive layer 130further provides flow channel 131. Desirable characteristics of the adhesive is that it provide sufficient adhesive strength between layers, that it be hydrophobic, and that it can be cleanly removed from a substrate. For example, in one embodiment the adhesive comprises a UV release adhesive having a high tack in the absence of UV light but has a low tack after exposure to UV light.

Preferably, the array is masked during UV light exposure. Thus, the substrate may be conveniently removed from the other chamber components following UV exposure and the array is easily scanned. In an optional embodiment, a microfluidic chamber has more than one adhesive layer for adhering of other components and devices to the microfluidic chamber as described below. Adhesives are optionally employed, as needed, to prevent evaporation from the microfluidic chamber. Alternatively, the membrane is directly adhered to the substrate by heating the edge of the membrane or the substrate surface, applying the membrane and the substrate surface together, and allowing them to cool.

By “mixer” and grammatical equivalents herein are meant a device configured to exert a force upon a microfluidic chamber or its contents, either directly or indirectly, such that the contents, usually liquid, of the chamber are mixed, as described below, For example, a mixer may be a shaker, a centrifuge, a circular mixer and the like (e.g. Innova 4080 rotary table top shaker, New Brunswick Scientific). In one embodiment, a force is applied by an object directly to a flexible membrane, such as a roller or wheel moved across a flexible membrane. In another embodiment, the mixer is a micro-disk as further described below. By “force” and grammatical equivalents herein are meant that which causes a motion or a change in motion of an object. Accordingly, a force with respect to a microfluidic device produces a motion or a change in motion of the contents of a microfluidic device but preferably is not sufficient to cause mechanical failure of the device. A force may be constant or variable. In a preferred embodiment a force is variable. By “variable force” and grammatical equivalents herein are meant a force that changes in magnitude, direction or duration with respect to the microfluidic chamber as a frame of reference. Accordingly, rotational, lateral, centrifugal, horizontal, vertical, pulsating forces and the like are contemplated by the present invention. The skilled artisan will appreciate that application of a variable force may be accomplished in many different ways without exceeding the scope of the present invention. For example, and not by way of any limitation, rotating the microfluidic chamber at varying speeds or about a point at varying radii.

In the embodiment of a microfluidic chamber comprising a flexible membrane, a mixer preferably is configured to deform the flexible membrane without causing mechanical failure of the membrane. In optional embodiments, the miter is configured to apply a force directly to the flexible membrane, the entire chamber, or the fluid contents of the chamber. For example, a microfluidic chamber may be affixed and shaken by a table top shaker. Without being bound by theory, the applied force produces elastic deformation of the flexible membrane thereby causing the contents of the chamber to be appropriately mixed. Alternatively, a force is directly applied to the liquid contents of a microfluidic chamber by a micro disk in fluidic communication with the chamber, as further described below.

In a preferred embodiment, when either a rigid or flexible membrane are used, a mixer preferably comprises a micro-disk in fluidic communication with the microfluidic chamber such that rotation of the micro-disk appropriately mixes the fluid contents of the chamber. The core of a micro-disk preferably comprises a “magnetic material” or “magnetizable material”. By “magnetic material” and grammatical equivalents herein are meant a substance that is susceptible to a magnetic field (e.g. iron, steel, magnets). Preferably, the core is encased within a material that is inert and does not physically or chemically react with the components of the fluid within the microfluidic chamber or contaminate the fluid and does not substantially shield the core from the magnetic field. For example, the core is preferably encased within a material comprising plastic (including acrylics, polystyrene and copolymers of styrene and other materials, polypropylene, polyethylene, polybutylene, polyimide, polycarbonate, polyurethanes, Teflon™, and derivatives thereof, etc.). Rotation of the micro-disk is coupled to an external magnetic field produced by a magnetic field generator (e.g. magnet, electromagnet). Without being bound by theory, the magnetic field is altered in a manner to cause the micro-disk to rotate, thereby causing mixing of the fluid contents of the microfluidic chamber. Accordingly, the micro-disk and magnetic field generator are in magnetic communication. Magnetic field generators generally fall into two categories: “on chip” and “off chip”; that is, for example, the generators can be contained within a microfluidic device itself, or they can be contained on an apparatus into which the device fits, such that proper alignment occurs between the micro-disk and the generator. The shape and size of the micro-disk are selected at the discretion of the practitioner. Preferably a micro-disk is tablet, disk, bar or discoid in shape. In a more preferred embodiment, a micro-disk comprises a disk about 8 mm by about 500 μm, preferably with with flanges. In practice, a micro-disk may be of any shape which results in mixing of the fluid within a microfluidic chamber.

In one embodiment, a surfactant is of a type and present at a concentration effective to substantially reduce nonspecific binding and promote mixing of sample fluid components within the chamber. Examples of surfactants include anionic surfactants (e.g. sodium, potassium, ammonium and lithium salts of lauryl sulfate), cationic surfactants, amphoteric surfactants, nonionic surfactants (e.g. polyethylene oxide, polyethylene oxides comprising an alkylphenol ethylene oxide condensate, TRITON® (Sigma Chemical Co.)). The surfactant concentration in the sample fluid is between about 0.1 wt. % and 10 wt. % of the sample fluid, preferably between about 0.5 wt. % and 5 wt. % of the sample fluid, more preferably between about 0.75 wt. % and 5 wt. % of the sample fluid; however, the exact concentration will vary with the surfactant selected, and those skilled in the art may readily optimize the concentration with respect to the desired results, i.e., reduction of nonspecific binding and facilitation of mixing within the sample fluid. Surfactants and their uses are further described in U.S. Pat. Nos. 6,287,850; 6,258,593, expressly incorporated by reference.

In one embodiment, mixing occurs in the.substantial absence of air or gas in the microfluidic chamber.

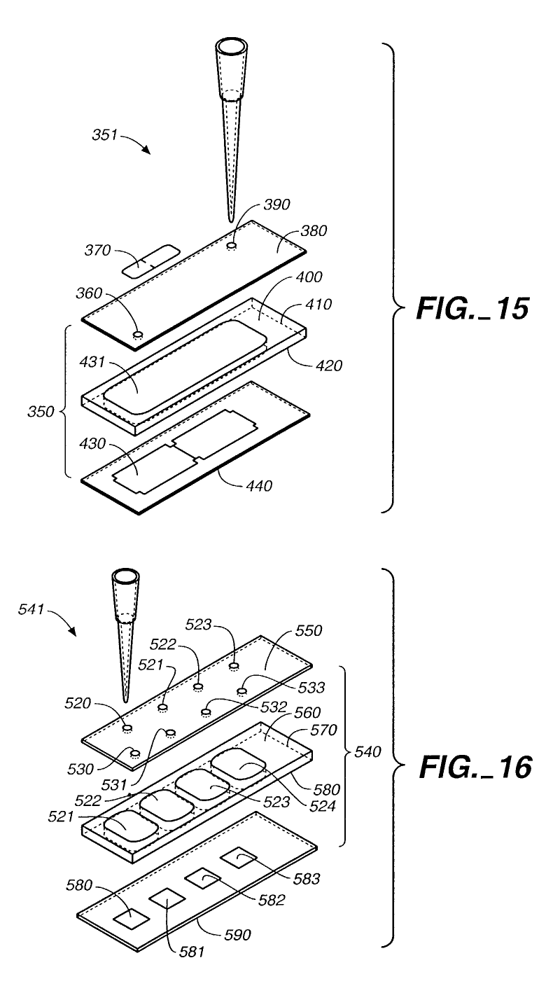

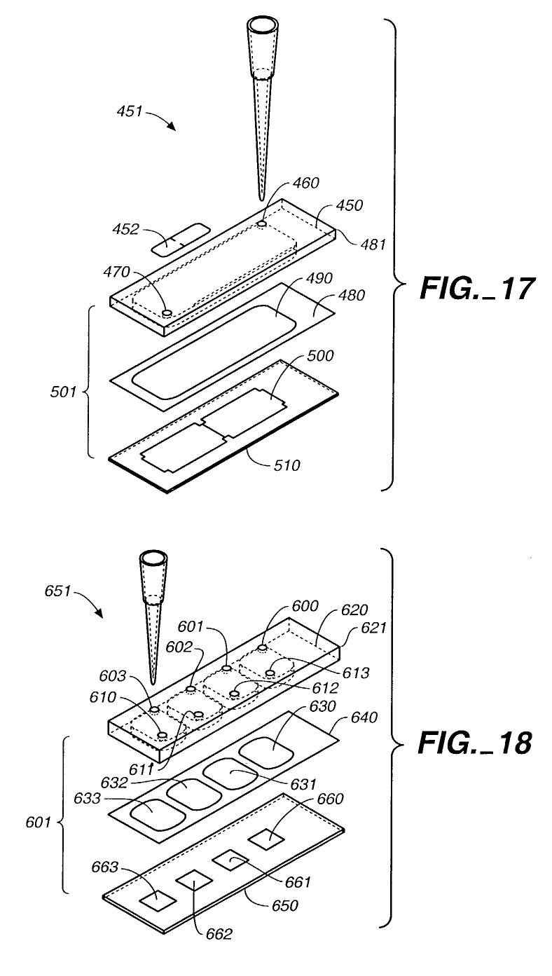

Alternatively, mixing occurs in the presence of air or gas, preferably inert, in the chamber. For example, a bubble in a microfluidic chamber is displaced by the application of a force to the chamber or its fluid contents. Without being bound by theory, movement of the bubble displaces the fluid resulting in mixing. Alternatively, a contiguous gap may be employed for mixing. Without being bound by theory, the contiguous gap permits displacement of the fluid within the chamber resulting in mixing. For example, FIG. 15 depicts one embodiment of a microfluidic chamber that employs a contiguous air gap between the sample fluid and the membrane. In FIG. 15, microfluidic chamber 350 defined by flat sheet membrane 380, spacer or perimeter shim 410 having void 431 therein, to which adhesives 410 and 420, are attached, and substrate 440 having array 430. Membrane 380 has ports 360 and 390 at opposite ends. Ports 360 and 390 are preferably sealed by an adhesive, such as tape 370 after sample fluid loading. The spacer or perimeter shim 410 including adhesives is preferably about 3.6 mm thick to allow a contiguous air pocket over the sample fluid over the entire array 430. The height of microfluidic chamber 350 is preferably about 0.38 inches. Shim 410 is preferably a low surface energy plastic, such as, polyolefin or PTFE, and the like, so that the target fluid does not wick up the wall of the shim. Alternatively, as depicted in FIGS. 16 and 18, perimeter shim 450 and 620, respectively are contiguous with the membrane and are constructed by injection molding techniques. To prevent drying of array 430 due to tilting of the chamber, the fluid thickness is preferably at least about 0.7 mm. Microfluidic chamber 310 is preferably held level with respect to gravity to within 1 degree for the “1-up” design shown in FIGS. 15 and 16 and to within 4 degrees for the “4-up” design shown in FIGS. 17 and 18 while stationary for at least about 1 minute. During mixing the tilt can be higher but preferably does not exceed an angle to prevent the sample fluid from re-wetting the internal surfaces of the chamber. Preferably, the substrate surface and the surface of the perimeter shim adjacent to the substrate are hydrophilic to promote fluid sample coverage of the array. Above the hydrophilic surface, the perimeter shim and membrane are preferably hydrophobic to inhibit sample fluid wetting of these areas.

**Anything to Add?

By “substrate”, “chip”, “biochip” and grammatical equivalents herein are meant any material that functions as a support for a membrane of a microfluidic chamber and is amenable to at least one method of the invention as further described below. Preferably, the substrate contains or can be modified to contain discrete individual sites for the attachment of binding molecules or binding ligands, e.g. capture probes, as further described below. As wil be appreciated by those in the art, the number of possible substrate compositions and the size and shape of the substrate is very large. Accordingly, in some embodiments the surface of the substrate is planar and in some embodiments the substrate may contain a cavity or have an irregular shape. The composition of the substrate will depend on a variety of factors, including the techniques used to create the substrate, the use of the substrate, the composition of the sample, sample possessing, the analyte to be detected, the size of internal structures, the presence or absence of electronic components, etc. Thus, in alternative embodiments, the substrate comprises channels, ports, ducts, valves, docking mechanisms, vias and the like to provide fluidic communication with other devices, chambers or modules; or to provide a means of access into and out of the chamber. Preferably, the substrate comprises a hydrophilic surface to promote sample fluid coverage of the array.

In a preferred embodiment, the substrate is made from a wide variety of materials including, but not limited to, silicon such as silicon wafers, silicon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdenum, tantalum, Kovar™, Kevlar™, Kapton™, Mylar™, brass, sapphire, etc. High quality glasses such as high melting borosilicate or fused silicas may be preferred for their UV transmission properties when any of the sample manipulation steps require light based technologies. Substrates of the present invention may be fabricated using a variety of techniques, including, but not limited to, hot embossing, such as described in H. Becker, et al, Sensors and Materials, 11, 297, (1999), hereby incorporated by reference, molding of elastomers, such as described in D. C. Duffy, et. al., Anal. Chem., 70, 4974, (1998), hereby incorporated by reference, injection molding, silicon fabrication and related thin film processing techniques, circuit board fabrication technology, and in a preferred embodiment, the substrates are fabricated using ceramic multilayer fabrication techniques, such as are outlined in PCT/US99/23324; U.S. Pat. No. 3,991,029; U.S. patent application Ser. Nos. 09/235,081; 09/337,086; 09/464,490; 09/492,013; 09/466,325; 09/460,281; 09/460,283; 09/387,691; 09/438,600; 09/506,178; 09/458,534; and Richard E. Mistier, “Tape Casting: The Basic Process for Meeting the Needs of the Electronics Industry,” Ceramic Bulletin, vol. 69, no. 6, pp. 1022-26 (1990); all of which are incorporated by reference in their entirety. In this embodiment, the substrates are made from layers of green-sheet that have been laminated and sintered together to form a substantially monolithic structure. Green-sheet is a composite material that includes inorganic particles of glass, glass-ceramic, ceramic, or mixtures thereof, dispersed in a polymer binder, and may also include additives such as plasticizers and dispersants. The green-sheet is preferably in the form of sheets that are 50 to 250 microns thick. The ceramic particles are typically metal oxides, such as aluminum oxide or zirconium oxide. An example of such a green-sheet comprising glass-ceramic particles is AX951 (E. I. Du Pont de Nemours and Co.). An example of a green-sheet that includes aluminum oxide particles is Ferro Alumina (Ferro Corp.). The composition of the green-sheet may also be custom formulated to meet particular applications. The green-sheet layers are laminated together and then fired to form a substantially monolithic multilayered structure.

Several advantages of using green-sheets include that various structures, for example, channels that provide fluidic communication, may be easily and accurately formed within the substrate, thereby permitting connections of the microfluidic device to other microfluidic processes on the same or another device. In another example, electrical connections may be easily formed within the substrate using thick-film paste (described in one or more of U.S. patent application Ser. Nos. 09/235,081; 09/337,086; 09/464,490; 09/492,013; 09/466,325; 09/460,281; 09/460,283; 09/387,691; 09/438,600; 09/506,178; and 09/458,534, expressly incorporated by reference in their entirety), which permits the integration of many microfluidic modules or processes (e.g. resistive heaters, pH sensors, temperature sensors, microwave lysis, microwave heating, electrical field lysis, PCR cycling, pumps (electrohydrodynamic or electroosmotic) and the like (see U.S. patent application Ser. No. 09/816,512 and PCT Application No. PCT/USO1/02664, both of which are expressly incorporated by reference in their entirety).

In one embodiment, the area of the substrate demarcated by the membrane comprises an array of binding molecules or binding ligands. Accordingly, the present invention provides array compositions comprising at least a first substrate with a surface comprising individual sites. By “array”, and “microarray” and grammatical equivalents herein are meant a plurality of binding binding molecules or binding ligands in an array format (e.g. a spatially addressable system). The size of the array will depend on the composition and end use of the array. In a preferred embodiment, the binding molecules are nucleic acids, for example, probes, capture probes, oligonucleotides and the like. Nucleic acids arrays are known in the art, and can be classified in a number of ways; both ordered arrays (e.g. the ability to resolve chemistries at discrete sites), and random arrays are included. Ordered arrays include, but are not limited to, those made using photolithography techniques (Affymetrix GeneChip™), spotting techniques (Synteni and others), printing techniques (Hewlett Packard and Rosetta), electrode arrays, three dimensional gel or gel pad arrays, etc.

The construction and use of solid phase nucleic acid arrays to detect target nucleic acids is well described in the literature. See, Fodor et al. (1991) Science, 251: 767-777; Sheldon et al. (1993) Clinical Chemistry 39(4): 718-719; Kozal et al. (1996) Nature Medicine 2(7): 753-759; Hubbell U.S. Pat. No. 5,571,639; and Pinkel et al. PCT/US95/16155 (WO 96/17958), incorporated by reference. In brief, a combinatorial strategy allows for the synthesis of arrays containing a large number of nucleic acid probes using a minimal number of synthetic steps. For instance, it is possible to synthesize and attach all possible DNA 8-mer oligonucleotides (48 or 65,536 possible combinations) using only 32 chemical synthetic steps. In general, very large scale immobilized polymer synthesis (VLSIPS) procedures provide a method of producing 4n different oligonucleotide probes on an array using only 4n synthetic steps.

Light-directed combinatorial synthesis of oligonucleotide arrays on a glass surface is performed with automated phosphoramidite chemistry and chip masking techniques similar to photoresist technologies in the computer chip industry. Typically, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

A 96-well automated multiplex oligonucleotide synthesizer (A.M.O.S.) has also been developed and is capable of making thousands of oligonucleotides (Lashkari et al. (1995) PNAS 93: 7912), incorporated by reference. Existing light-directed synthesis technology can generate high-density arrays containing- over 65,000 oligonucleotides (Lipshutz et al. (1995) BioTech. 19: 442), incorporated by reference.

Combinatorial synthesis of different oligonucleotide analogues at different locations on the array is determined by the pattern of illumination during synthesis and the order of addition of coupling reagents. Monitoring of hybridization of target nucleic acids to the array is typically performed with fluorescence microscopes or laser scanning microscopes. In addition to being able to design, build and use probe arrays using available techniques, one of skill in the art is also able to order custom-made arrays and array-reading devices from manufacturers specializing in array manufacture. For example, Affymetrix Corp. (Santa Clara, Calif.) manufactures DNA VLSIP arrays.

It will be appreciated that oligonucleotide design is influenced by the intended application. For example, where several oligonucleotide -tag interactions are to be detected in a single assay, e.g., on a single DNA chip, it is desirable to have similar melting temperatures for all of the probes. Accordingly, the length of the probes are adjusted so that the melting temperatures for all of the probes on the array are closely similar (it will be appreciated that different lengths for different probes may be needed to achieve a particular Tₘ where different probes have different GC contents). Although melting temperature is a primary consideration in probe design, other factors are optionally used to further adjust probe construction, such as selecting against primer self-complementarity and the like.

In a preferred embodiment CodeLink™ array technology is used, CodeLink™ technology provides an apparatus for performing high-capacity biological reactions on a biochip substrate having an array of binding sites. It provides a hybridization chamber having one or more arrays, preferably comprising arrays consisting of hydrophilic, 3-dimensional gel and most preferably comprising arrays consisting of 3-dimensional polyacrylamide gels, wherein nucleic acid hybridization is performed by reacting a biological sample containing a target molecule of interest with a complementary oligonucleotide probe immobilized on the gel. Nucleic acid hybridization assays are advantageously performed using probe array technology, which preferably utilizes binding of target single-stranded DNA onto immobilized oligonucleotide probes. Preferred arrays include those outlined in U.S. patent application Ser. Nos. 09/458,501, 09/459,685, 09/464,490, 09/605,766, PCT/US00/34145, 09/492,013, PCT/US01/02664, WO 01/54814, 09/458,533, 09/344,217, PCT/US99/27783, 09/439,889, PCT/US00/42053 and WO 01/34292, all of which are hereby incorporated by reference in their entirety.

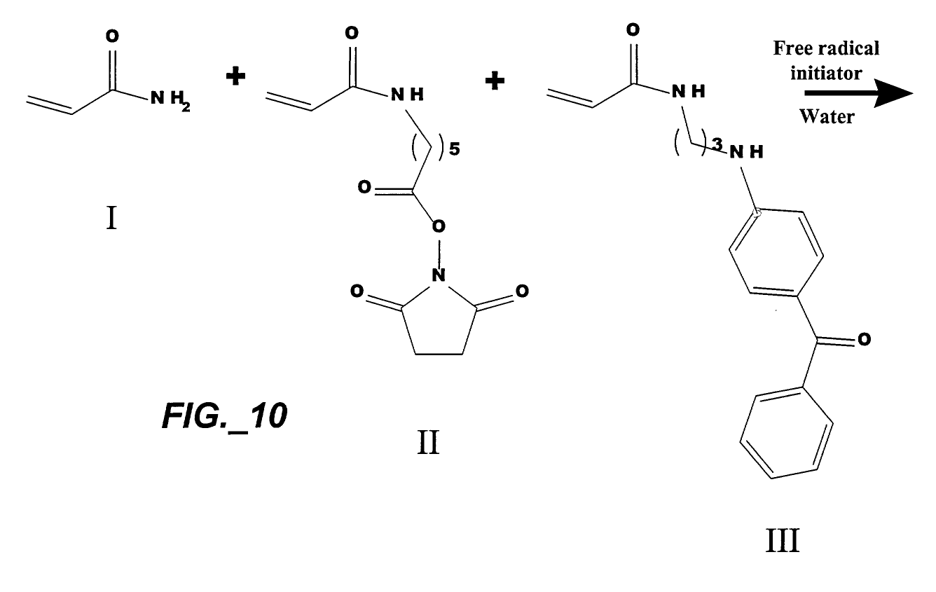

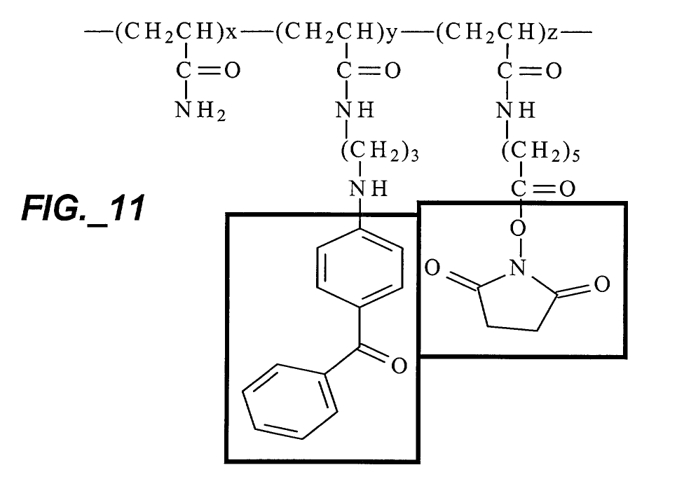

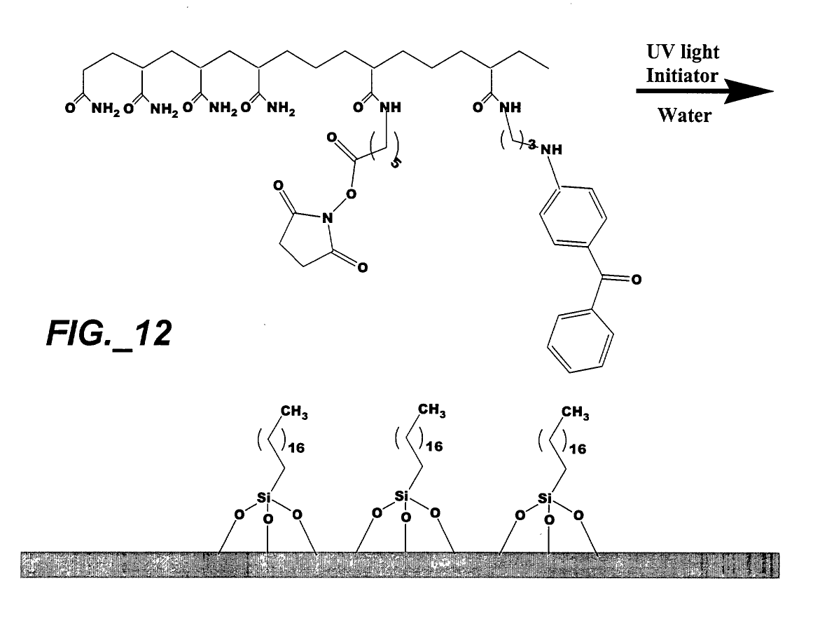

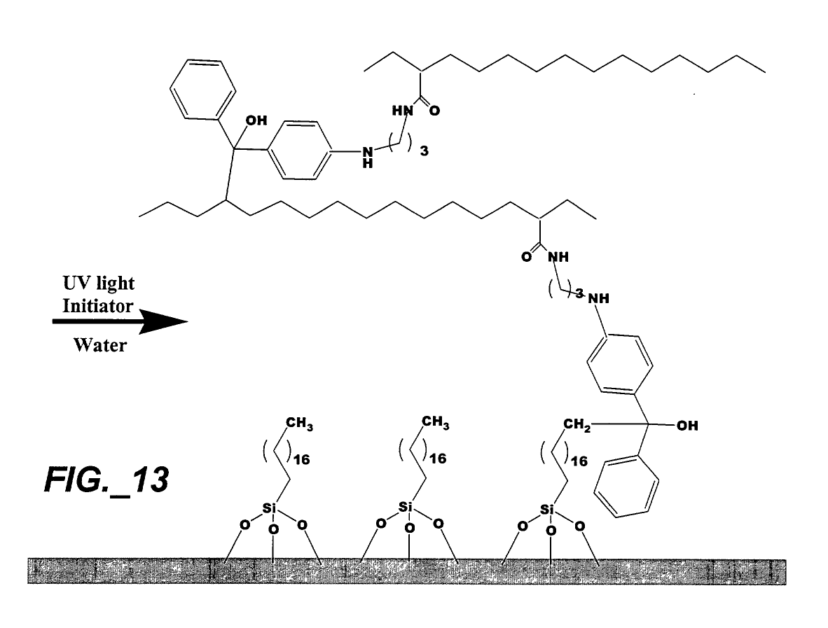

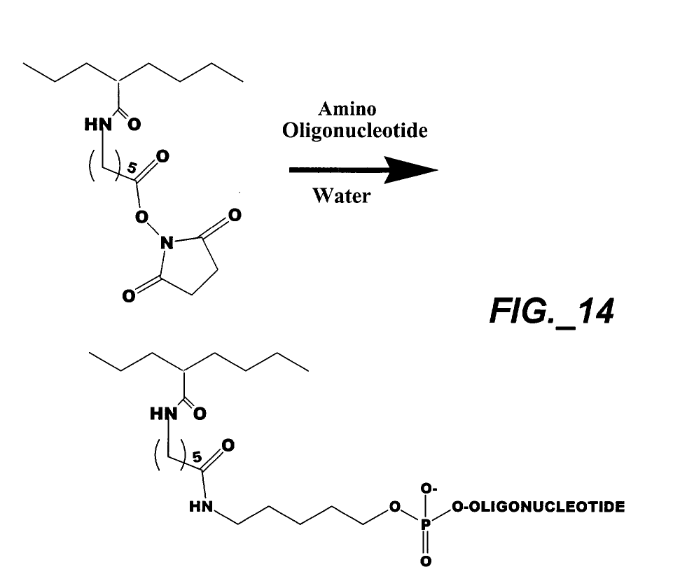

The preparation of CodeLink™ arrays is described in U.S. Pat. Nos. 5,002,582; 5,512,329; 5,714,360; and 5,741,551; and EP 0 326 579 B1, all of which are incorporated by reference. In a preferred embodiment, a gel polymer is synthesized having different functionalities. As shown in FIGS. 10 and 11, a gel polymer having a crosslinking agent for attachment to a substrate and an oligonucleotide attachment agent is synthesized by the co-polymerization of acrylamide, acrylamide-NHS ester, and acrylamide-benzophenone in the presence of a free radical initiator, such as, dibenzoyl peroxide. The benzophenone is a photoactive ketone that covalently attaches to a methyl group of the silanized glass substrate via the carbonyl under UV light (FIGS. 12-13). Under these conditions, the carbonyl of the benzophenone is highly reactive, and therefore results in a higher cross linked three dimensional structure (FIG. 13) which provides an increased number of oligonucleotide binding sites at each site of bound polymer. Oligonucleotides having an amine modified 3′ or 5′ terminus for attachment to the gel polymer are desalted to remove amine contaminants and purified by ethanol precipitation or column chromatography. The purified amino-oligo is adjusted to a final concentration of about 10-25 nmole/ml in 150 mM sodium phosphate, pH 8.5. For amino-oligos from about 0.1 to 1.0 Kb the adjusted concentration to about 0.1-0.5 mg/ml in 150 mM sodium phosphate, pH 8.5. The amino-oligo solution is spotted on the modified slides to form microarrays (FIG. 14). The slides are incubated in a storage box inside a saturated NaCl humidification chamber for about 4 to 72 hours before use.

As those in the art will appreciate, the size of the array will vary. Arrays containing from about 2 different capture probes to many millions can be made, with very large arrays being possible. Preferred arrays generally range from about 25 different capture probes to about 100,000, depending on array composition, with array densities varying accordingly. In a preferred embodiment, capture probes are only attached at one end, either 3′ or 5′ end.

Generally, the capture probe allows the attachment of a target analyte to the array for the purposes of detection. As is more fully outlined below, attachment of the target analyte to the capture probe may be direct (i.e. the target sequence binds to the capture probe) or indirect (one or more capture extender ligands may be used).

By “capture binding ligand”, “capture binding partner” and grammatical equivalents herein are meant a compound that is used to bind a component of the sample. Suitable binding moieties will depend on the sample component to be isolated or removed either a contaminant (for removal) or the target analyte (for enrichment). In some embodiments, as outlined below, the binding ligand is used to probe for the presence of the target analyte, and that will bind to the analyte.

As will be appreciated by those in the art, the composition of the binding ligand will depend on the sample component to be separated. Binding ligands for a wide variety of analytes are known or can be readily found using known techniques. For example, when the component is a protein, the binding ligands include proteins (particularly including antibodies or fragments thereof (FAbs, etc.)) or small molecules. When the sample component is a metal ion, the binding ligand generally comprises traditional metal ion ligands or chelators. Preferred binding ligand proteins include peptides. For example, when the component is an enzyme, suitable binding ligands include substrates and inhibitors. Antigen-antibody pairs, receptor-ligands, and carbohydrates and their binding partners are also suitable component-binding ligand pairs. The binding ligand may be nucleic acid, when nucleic acid binding proteins are the targets; alternatively, as is generally described in U.S. Pat. Nos. 5,270,163, 5,475,096, 5,567,588, 5,595,877, 5,637,459, 5,683,867, 5,705,337, and related patents, hereby incorporated by reference, nucleic acid “aptomers” can be developed for binding to virtually any target analyte. Similarly, there is a wide body of literature relating to the development of binding partners based on combinatorial chemistry methods. In this embodiment, when the binding ligand is a nucleic acid, preferred compositions and techniques are outlined in PCT US97/20014, hereby incorporated by reference.

In a preferred embodiment, the binding of the sample component to the binding ligand is specific, and the binding ligand is part of a binding pair. By “specifically bind” herein is meant that the ligand binds the component, for example the target analyte, with specificity sufficient to differentiate between the analyte and other components or contaminants of the test sample. The binding should be sufficient to remain bound under the conditions of the separation step or assay, including wash steps to remove non-specific binding. In some embodiments, for example in the detection of certain biomolecules, the disassociation constants of the analyte to the binding ligand will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

As will be appreciated by those in the art, the composition of the bindingligand will depend on the composition of the target analyte. Binding ligands to a wide variety of analytes are known or can be readily found using known techniques. For example, when the analyte is a single-stranded nucleic acid, the binding ligand is generally a substantially complementary nucleic acid. Similarly the analyte may be a nucleic acid binding protein and the capture binding ligand is either a single-stranded or double-stranded nucleic acid; alternatively, the binding ligand may be a nucleic acid binding protein when the analyte is a single or double-stranded nucleic acid. When the analyte is a protein, the binding ligands include proteins or small molecules. Preferred binding ligand proteins include peptides. For example, when the analyte is an enzyme, suitable binding ligands include substrates, inhibitors, and other proteins that bind the enzyme, i.e. components of a multi-enzyme (or protein) complex. As will be appreciated by those in the art, any two molecules that will associate, preferably specifically, may be used, either as the analyte or the binding ligand. Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. These may be wild-type or derivative sequences. In a preferred embodiment, the binding ligands are portions (particularly the extracellular portions) of cell surface receptors that are known to multimerize, such as the growth hormone receptor, glucose transporters (particularly GLUT4 receptor), transferrin receptor, epidermal growth factor receptor, low density lipoprotein receptor, high density lipoprotein receptor, leptin receptor, interleukin receptors including IL-1, IL-2, IL-3, IL-4, IL-5, IL-6, IL-7, IL-8, IL9, IL-11, IL-12, IL-13, IL-15 and IL-17 receptors, VEGF receptor, PDGF receptor, EPO receptor, TPO receptor, ciliary neurotrophic factor receptor, prolactin receptor, and T-cell receptors.

When the sample component bound by the binding ligand is the target analyte, it may be released for detection purposes if necessary, using any number of known techniques, depending on the strength of the binding interaction, including changes in pH, salt concentration, temperature, etc. or the addition of competing ligands, etc.

In a preferred embodiment, the capture binding ligand is a nucleic acid, sometimes referred to herein as a “capture probe”. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al, Eur. J. Biochem. 81:579 (1977); Letsinger et al., Nucl. Acids Res. 14:3487 (1986); Sawai et al., Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023; 5,637,684; 5,602,240; 5,216,141; and 4,469,863; Kiedrowshi et al, Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp. 169-176). Several nucleic acid analogs are described in Rawls, C & E News Jun. 2, 1997 page 35. Nucleic acid analogs also include “locked nucleic acids”. All of these references are hereby expressly incorporated by reference. These modifications of the ribose-phosphate backbone may be done to facilitate the addition of labels, or to increase the stability and half-life of such molecules in physiological environments.

As will be appreciated by those in the art, all of these nucleic acid analogs may find use in the present invention. In addition, mixtures of naturally occurring nucleic acids and analogs may be used. Alternatively, mixtures of different nucleic acid analogs, and mixtures of naturally occurring nucleic acids and analogs may be used.

As outlined herein, the nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as nucleosides.

In a preferred embodiment, the capture binding ligand is a protein. By “proteins” and grammatical equivalents herein are meant proteins, oligopeptides and peptides, derivatives and analogs, including proteins containing non-naturally occurring amino acids and amino acid analogs, and peptidomimetic structures. The side chains may be in either the (R) or (S) configuration. In a preferred embodiment, the amino acids are in the (S) or L-configuration. As discussed below, when the protein is used as a binding ligand, it may be desirable to utilize protein analogs to retard degradation by sample contaminants.

In a preferred embodiment, the devices as described herein, for example, chambers, channels, membranes, substrates, tubing, ports, ducts, valves, docking mechanisms, vias, modules etc. are made from, or coated with, biocompatible materials as needed in regions where they will come into contact with biological samples to reduce non-specific binding, to allow the attachment of binding ligands, for biocompatibility, for flow regulation, etc. In particular, materials that provide a surface that retards the non-specific binding of biomolecules, e.g. a “non sticky” surface, are preferred. For example, when a chamber is used for PCR or amplification reactions a “non sticky” surface prevents enzymatic components of the reaction mixture from sticking to the surface and being unavailable in the reaction. In other embodiments, biocompatible materials do not introduce contaminant analytes into sample fluids.

Biocompatible materials include, but are not limited to, plastic (including acrylics, polystyrene and copolymers of styrene and other materials, polypropylene, polyethylene, polybutylene, polyimide, polycarbonate, polyurethanes, Teflon™, and derivatives thereof, etc.) Other materials include combinations of plastic and printed circuit board (PCB; defined below). For example at least one side of a chamber is printed circuit board, while one or more sides of a chamber are made from plastic. In a preferred embodiment, three sides of a chamber are made from plastic and one side is made from printed circuit board. In addition, the chambers, channels, and other components of the systems described herein may be coated with a variety of materials to reduce non-specific binding. These include proteins such as caseins and albumins (bovine serum albumin, human serum albumin, etc.), parylene, other polymers, etc.

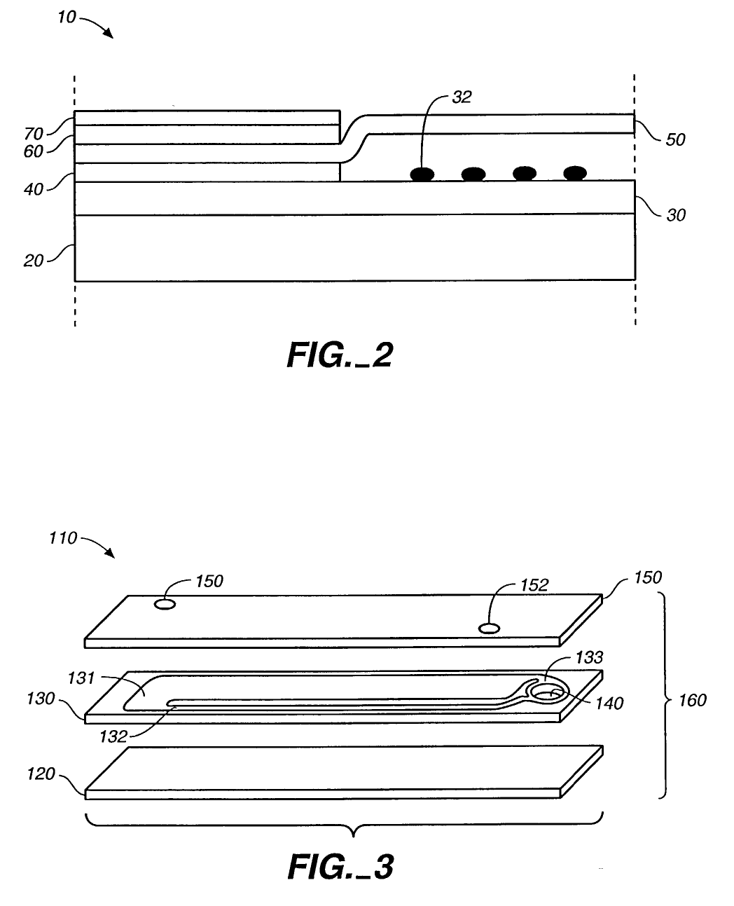

Microfluidic systems of the present invention may be configured in a large variety of ways to perform a wide variety of applications or functions. Generally, a microfluidic system comprises at least one microfluidic chamber and a mixer, as described above. In other embodiments, a microfluidic system may comprise any number of microfluidic chambers and mixers as selected at the discretion of the practitioner. For devices comprising more than one microfluidic chamber, each chamber optionally is self-contained or in fluidic communication with another system component. In another embodiment, the functions of the microfluidic chambers may be the same or different. The physical arrangement of the chambers is selected at the discretion of the practitioner. For example, the chambers are optionally arranged in series or parallel or combinations thereof. They may be arranged linearly or in the same plane or stacked. Examples of configurations of the microfluidic systems are depicted in FIGS. 1-5. See for example U.S. Pat. No. 5,603,351, PCT US96/17116, and “Multilayered Microfluidic Devices For Analyte Reactions” filed in the PCT Dec. 11, 2000, Serial No. PCT/US00/33499, hereby incorporated by reference. Additional examples of microfluidic systems are depicted in FIGS. 1-5.

In a preferred embodiment, the microfluidic systems or chambers are in fluidic communication and each provide a certain functionality. Thus, the microfluidic systems and chambers of the present invention find use as modules, for example, in sample collection, cell handling (for example, for cell lysis, cell removal, cell separation or capture, cell growth, etc.), reagent mixing; separation, for example, for electrophoresis, gel filtration, ion exchange/affinity chromatography (capture and release) etc.; reaction modules for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), and nucleic acid sequence based amplification (NASBA)), chemical, physical or enzymatic cleavage or alteration of the target analyte, or chemical modification of the target analyte; thermal modules for heating and cooling (which may be part of other modules, such as reaction modules); storage modules for assay reagents; and detection modules, as further described below and in WO00/62931, hereby incorporated by reference.

“Fluidic communication” and grammatical equivalents herein are intended to describe a means for the transfer or flow of a fluid between modules or components of a microfluidic device. For example, microfluidic chambers are in fluidic communication when connected by a channel or the like through which a fluid is transferred from one chamber to the other. In another example, a micro-disk is in fluidic communication with a microfluidic chamber such that rotation of the micro-disk mixes the fluid content of a chamber. Accordingly, the micro-disk may be within the microfluidic chamber or in separate housing and connected by one or more channels to one or more chambers in series or in parallel, as described below. As will be appreciated by the skilled artist, channels, tubing, ports, ducts, valves, docking mechanisms, vias, pumps and the like are contemplated to provide fluidic communication.

By “channel”, “microchannel” and grammatical equivalents herein are generally meant a region designed to have fluid moved through it, substantially from one end of the channel to another. Accordingly, channels are an example of a device that provides fluidic communication. In some embodiments, channels are designed to allow fluid to come into contact with an electrode, a physical constriction or a detection module, as described further below. A channel may have any shape, for example, it may be linear, serpentine, arc shaped and the like. The cross-sectional dimension of the channel may be square, rectangular, semicircular, circular, etc.

Additionally, the cross-sectional dimension of the channel may change across its length. Channels may be closed and completely internal to the device, or they may be substantially open to accommodate the introduction or removal of sample or agents. The channels have preferred depths on the order of 0.1 μm to 100 μm, typically 2-50 μm. The channels have preferred widths on the order of 2.0 to 500 μm, more preferably 3-100 μm. For many applications, channels of 5-50 μm are useful. In one embodiment, a channel with a 200 μm cross-section is provided. There may be multiple and interconnected channels. In one embodiment of the present invention, channels in one orientation intersect at multiple locations with channels having an orthogonal orientation.

In a preferred embodiment, the microfluidic devices of the invention include at least one fluid pump. Pumps generally fall into two categories: “on chip” and “off chip”; that is, the pumps (generally electrode based pumps) can be contained within a microfluidic device itself, or they can be contained on an apparatus into which the device fits, such that alignment occurs of the required flow channels to allow pumping of fluids.

In a preferred embodiment, the pumps are contained on the device itself. These pumps are generally electrode based pumps; that is, the application of electric fields can be used to move both charged particles and bulk solvent, depending on the composition of the sample and of the device. Suitable “on device” pumps include, but are not limited to, electroosmotic (EO) pumps and electrohydrodynamic (EHD) pumps; these electrode based pumps have sometimes been referred to in the art as “electrokinetic (EK) pumps”. All of these pumps rely on configurations of electrodes placed along a flow channel to result in the pumping of the fluids comprising the sample components. As is described in the art, the configurations for each of these electrode based pumps are slightly different; for example, the effectiveness of an EHD pump depends on the spacing between the two electrodes, with the closer together they are, the smaller the voltage required to be applied to effect fluid flow. Alternatively, for EO pumps, the spacing between the electrodes should be larger, with up to one-half the length of the channel in which fluids are being moved, since the electrodes are only involved in applying force, and not, as in EHD, in creating charges on which the force will act.

In a preferred embodiment, an electroosmotic pump is used. Electroosmosis (EO) is based on the fact that the surface of many solids, including quartz, glass and others, become variously charged, negatively or positively, in the presence of ionic materials. The charged surfaces will attract oppositely charged counterions in aqueous solutions. Applying a voltage results in a migration of the counterions to the oppositely charged electrode, and moves the bulk of the fluid as well. The volume flow rate is proportional to the current, and the volume flow generated in the fluid is also proportional to the applied voltage. Electroosmostic flow is useful for liquids having some conductivity and generally not applicable for non-polar solvents. EO pumps are described in U.S. Pat. Nos. 4,908,112 and 5,632,876, PCT US95/14586 and WO97/43629, incorporated by reference.

In a preferred embodiment, an electrohydrodynamic (EHD) pump is used. In EHD, electrodes in contact with the fluid transfer charge when a voltage is applied. This charge transfer occurs either by transfer or removal of an electron to or from the fluid, such that liquid flow occurs in the direction from the charging electrode to the oppositely charged electrode. EHD pumps can be used to pump resistive fluids such as non-polar solvents. EHD pumps are described in U.S. Pat. No. 5,632,876, hereby incorporated by reference.

The electrodes of the pumps preferably have a diameter from about 25 microns to about 100 microns, more preferably from about 50 microns to about 75 microns. Preferably, the electrodes protrude from the top of a flow channel to a depth of from about 5% to about 95% of the depth of the channel, with from about 25% to about 50% being preferred. In addition, as described in PCT US95/14586, incorporated by reference, an electrode-based internal pumping system can be integrated into the liquid distribution system of the devices of the invention with flow-rate control at multiple pump sites and with fewer complex electronics if the pumps are operated by applying pulsed voltages across the electrodes; this gives the additional advantage of ease of integration into high density systems, reductions in the amount of electrolysis that occurs at electrodes, reductions in thermal convection near the electrodes, and the ability to use simpler drivers, and the ability to use both simple and complex pulse wave geometries.

The voltages required to be applied to the electrodes to cause fluid flow depends on the geometry of the electrodes and the properties of the fluids to be moved. The flow rate of the fluids is a function of the amplitude of the applied voltage between electrodes, the electrode geometry and the fluid properties, which can be easily determined for each fluid. Test voltages used may be up to about 1500 volts, but an operating voltage of about 40 to 300 volts is desirable. An analog driver is generally used to vary the voltage applied to the pump from a DC power source. A transfer function for each fluid is determined experimentally as that applied voltage that produces the desired flow or fluid pressure to the fluid being moved in the channel. However, an analog driver is generally required for each pump along the channel and is suitable as an operational amplifier.

In a preferred embodiment, a micromechanical pump is used, either “on-” or “off-chip”, as is known in the art.

In a preferred embodiment, one or more pumps are used to transport target analytes to a detection module. In another embodiment, one or more pumps are used to contact a module with a sample or an agent, as described below. In other embodiments, pumps are used to agitate a sample or wash contaminant analytes from a concentration module, as described below.

In a preferred embodiment, the microfluidic devices of the invention include at least one fluid valve that can control the flow of fluid into or out of a module of the device, or divert the flow into one or more channels. A variety of valves are known in the art. For example, in one embodiment, the valve may comprise a capillary barrier, as generally described in PCT US97/07880, incorporated herein by reference. In this embodiment, the channel opens into a larger space designed to favor the formation of an energy minimizing liquid surface such as a meniscus at the opening. Preferably, capillary barriers include a dam that raises the vertical height of a channel immediately before the opening into a larger space such as a chamber. In addition, as described in U.S. Pat. No. 5,858,195, incorporated herein by reference, a type of “virtual valve” can be used.

In a preferred embodiment, the microfluidic devices of the invention include one or more valves controlling the flow of fluids into and out of the chamber. The number of valves in the cartridge depends on the number of channels and chambers, and the desired application. In some embodiments, the microfluidic device is designed to include one or more loading ports or valves that can be closed off or sealed after the sample is loaded. It is also possible to have multiple loading ports into a single chamber; for example, a first port is used to load sample and a second port is used to add reagents. In these embodiments, the microfluidic device may have a vent. The vent can be configured in a variety of ways. In some embodiments, the vent can be a separate port, optionally with a valve, that leads out of the microfluidic chamber. Alternatively, the vent may be a loop structure that vents liquid and/or air back into the inlet port.

In a preferred embodiment, the microfluidic devices of the invention include a port, such as inlet or outlet ports, or vents. “Inlet and outlet port”, “port” and grammatical equivalents as used herein refer to one or more openings in a microfluidic device suitable for introducing a sample or other fluid into a channel or chamber or removing a sample, waste, or other fluid. Optionally, a septum in each port provides a sealing mechanism against a pipet tip or other device and automatically closing to limit evaporation from the chamber. Septa can be assembled into the port or injection molded into the port. “Vent”, as discussed above, generally refers to an opening in a microfluidic device for pressure equalization. In one embodiment, the ports are designed for use with conventional pipettes. In another embodiment, multiple inlet ports are provided for the introduction of a variety of fluids, including lysing agents, amplification agents, or sample fluid containing target analytes.

Ports may optionally comprise a seal to prevent or reduce the evaporation of the sample or agents from a chamber. In a preferred embodiment, the seal comprises a gasket, or valve through which a pipette or syringe can be pushed. The gasket or valve Gan be rubber or silicone or other suitable materials, such as materials containing cellulose. In another embodiment the seal can be reversibly removed, such as, a piece of tape.

In another embodiment, the microfluidic devices comprises channels or chambers that are substantially open. For example, a channels or chambers having rectangular cross-section may have only three walls. In this embodiment, then, the “inlet port” is the top of the device itself, and may subsequently be sealed with another material comprising the fourth wall of the channels or chambers, or another material, such as mineral oil.

Microfluidic systems and chambers as used herein may optionally include devices using one or more component to influence or monitor the temperature of a sample, referred to generally as a “thermal module”. For example, heaters, including thin-film resistive heating elements, may be provided “on-” or “off-chip”. Similarly, coolers, such as heat sinks or heat exchange conduits, may be provided “on-” or “off-chip”. Temperature monitoring devices may similarly be incorporated “on-” or “off-chip” and are known in the art. The composition and design of heaters, coolers, and temperature monitors will be dictated by the application and the material composition of the microfluidic device.

In one embodiment, heaters, coolers, and temperature monitors are provided to achieve thermal cycling of a chamber to perform PCR.

Suitable thermal modules are described in U.S. Pat. Nos. 5,498,392 and 5,587,128, and WO 97/16561, incorporated by reference, and may comprise electrical resistance heaters, pulsed lasers or other sources of electromagnetic energy directed to the microfluidic device. It should also be noted that when heating elements are used, it may be desirable to have a chamber be relatively shallow, to facilitate heat transfer; see U.S. Pat. No. 5,587,128, incorporated by reference.

When the devices of the invention include thermal modules, preferred embodiments utilize microfluidic devices fabricated to have low thermal conductivity in order to minimize thermal crosstalk between adjacent chambers, which permits independent thermal control of each chamber or channel.

In certain embodiments, the temperature of a device is increased using a thermal module comprising an integrated heater. In preferred embodiments, the integrated heater is a resistive heater, and more preferably a thick film resistive heater plate. Alternatively, channels, chambers and other component devices can be heated through the use of metal lines integrated beneath the well or surrounding sides of the chambers, channels etc, more preferably in a coil having one or more loops, in vertical or horizontal orientation. Parallel, variable heating of individual chambers or channels in a microchip array may be accomplished through the use of addressing schemes, preferably a column-and-row or individual electrical addressing scheme, in order to independently control the heat output of the resistive heaters in the vicinity of each chamber or channel.

In certain embodiments, the temperature of the device is decreased using a thermal module comprising an integrated cooler. In preferred embodiments, the integrated cooler is a metal via at the bottom of each chamber or channel. In further preferred embodiments, the integrated cooler is a thermoelectric cooler attached to or integrated into the substrate beneath each chamber or channel. Optionally, a metal via is in thermal contact with a metal plate, an array of metal discs or a thermoelectric cooler, each of which functions as a heat sink or an active cooling means. Commercially-available thermo-electric coolers can also be incorporated into the inventive apparatus, because they can be obtained in a wide range of dimensions, including components of a size required for the fabrication of the microfluidic devices of the present invention. In embodiments comprising metal heat sinks encompassing a metal plate or an array of metal discs, the plate or discs are composed of iron, aluminum, or other suitable metal. Parallel, variable cooling of individual chambers or channels in a microfluidic device may be accomplished through the use of addressing schemes, preferably a column-and-row or individual electrical addressing scheme, in order to independently control heat dissipation using cooling elements in the vicinity of each chamber or channel.

In preferred embodiments of the microfluidic devices of the invention, the thermal module includes temperature monitors, to monitor the temperature of the chamber or channel using an integrated resistive thermal detector or a thermocouple. This can be incorporated into the substrate or added later, and is in thermal contact and proximity to the chamber or channel structures of the microfluidic devices of the invention. The resistive thermal detector can be fabricated from a commercially available paste that can be processed in a customized manner for any given design. Such thermocouples are commercially available in sizes of at least 250 microns, including the sheath. In certain alternative embodiments, the temperature of the chambers or channels is monitored using an integrated optical system, for example, an infrared-based system.

In a preferred embodiment, the devices of the invention include a cell handling module. This is of particular use when the sample comprises cells that either contain the target analyte or that must be removed in order to detect the target analyte. Thus, for example, the detection of particular antibodies in blood can require the removal of the blood cells for efficient analysis, or the cells (and/or nucleus) must be lysed prior to detection. In this context, “cells” include eukaryotic and prokaryotic cells, and viral particles-that may require treatment prior to analysis, such as the release of nucleic acid from a viral particle prior to detection of target sequences. In addition, cell handling modules may also utilize a downstream means for determining the presence or absence of cells. Suitable cell handling modules include, butare not limited to, cell lysis modules, cell removal modules, and cell separation or capture modules. In addition, as for all the modules of the invention, the cell handling module may be integrated with other modules, or independent and in fluidic communication, or capable of being brought into fluidic communication, via a channel or the like with at least one other module of the invention.

In a preferred embodiment, the cell handling module includes a cell lysis module. As is known in the art, cells may be lysed in a variety of ways, depending on the cell type. In one embodiment, as described in EP 0 637 998 B1 and U.S. Pat. No. 5,635,358, hereby incorporated by reference, the cell lysis module may comprise cell membrane piercing protrusions that extend from a surface of the cell handling module. As fluid is forced through the device, the cells are ruptured. Similarly, this may be accomplished using sharp edged particles trapped within a cell handling chamber. Alternatively, the cell lysis module can comprise a region of restricted cross-sectional dimension, which results in cell lysis upon pressure. In a preferred embodiment, the cell lysis module comprises a concentration module, described below, that concentrates and traps the cells in a physical constriction. As the cells are trapped at the physical constriction, lysing agent is applied to the area of the physical constriction, causing lysis. In another preferred embodiment, localized heating causes cell lysis as the cells are trapped at a physical constriction, or other area of maximum or minimum electric field strength.

In a preferred embodiment, the cell lysis module comprises a cell lysing agent, such as guanidium chloride, chaotropic salts, enzymes, such as lysozymes, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample.

The cell lysis module may also include, either internally or externally, a filtering module for the removal of cellular debris as needed. This filter may be microfabricated between the cell lysis module and the subsequent module to enable the removal of the lysed cell membrane and other cellular debris components; examples of suitable filters are shown in EP 0 637 998 B1, incorporated by reference.

In a preferred embodiment, the cell handling module includes a cell separation or capture module. This embodiment utilizes a cell capture region comprising binding sites capable of reversibly binding a cell surface molecule to enable the selective isolation (or removal) of a particular type of cell from the sample population, for example, white blood cells for the analysis of chromosomal nucleic acid, or subsets of white blood cells. These binding moieties may be immobilized either on the surface of the module or on a particle trapped within the module (e.g. a bead) by physical absorption or by covalent attachment. Suitable binding moieties will depend on the cell type to be isolated or removed, and generally includes antibodies and other binding ligands, such as ligands for cell surface receptors, etc. Thus, a particular cell type may be removed from a sample prior to further handling, or the assay is designed to specifically bind the desired cell type, wash away the non-desirable cell types, followed by either release of the bound cells by the addition of reagents or solvents, physical removal (e.g. higher flow rates or pressures), or even in situ lysis.

Alternatively, a cellular “sieve” can be used to separate cells on the basis of size. This can be done in a variety of ways, including protrusions from the surface that allow size exclusion, a series of narrowing channels, a weir, or a diafiltration type setup.

In a preferred embodiment, the cell handling module includes a cell removal module. This may be used when the sample contains cells that are not required in the assay or are undesirable. Generally, cell removal will be done on the basis of size exclusion as for “sieving”, above, with channels exiting the cell handling module that are too small for the cells.

In a preferred embodiment, the cell handling module includes a cell concentration module. As will be appreciated by those in the art, this is done using “sieving” methods, for example to concentrate the cells from a large volume of sample fluid prior to lysis.

In a preferred embodiment, the devices of the invention include a separation module. Separation in this context means that at least one component of the sample is separated from other components of the sample. This can comprise the separation or isolation of the target analyte, or the removal of contaminants that interfere with the analysis of the target analyte, depending on the assay.

In a preferred embodiment, the separation module includes chromatographic-type separation media such as absorptive phase materials, including, but not limited to reverse phase materials (e.g. C₈ or C₁₈ coated particles, etc.), ion-exchange materials, affinity chromatography materials such as binding ligands, etc. See U.S. Pat. No. 5,770,029, herein incorporated by reference.

In a preferred embodiment, the separation module utilizes binding ligands, as is generally outlined herein for cell separation or analyte detection. In this embodiment, binding ligands are immobilized (again, either by physical absorption or covalent attachment, as described above) within the separation module (again, either on the internal surface of the module, on a particle such as a bead, filament or capillary trapped within the module, for example through the use of a frit). Suitable binding moieties will depend on the sample component to be isolated or removed. By “binding ligand” or grammatical equivalents herein is meant a compound that is used to bind a component of the sample, either a contaminant (for removal) or the target analyte (for enrichment). In some embodiments, as outlined below, the binding ligand is used to probe for the presence of the target analyte, and that will bind to the analyte.

In a preferred embodiment, the separation module includes an electrophoresis module, as is generally described in U.S. Pat. Nos. 5,770,029; 5,126,022; 5,631,337; 5,569,364; 5,750,015, and 5,135,627, all of which are hereby incorporated by reference. In electrophoresis, molecules are primarily separated by different electrophoretic mobilities caused by their different molecular size, shape and/or charge. Microcapillary tubes have recently been used for use in microcapillary gel electrophoresis (high performance capillary electrophoresis (HPCE)). One advantage of HPCE is that the heat resulting from the applied electric field is efficiently dissipated due to the high surface area, thus allowing fast separation. The electrophoresis module serves to separate sample components by the application of an electric field, with the movement of the sample components being due either to their charge or, depending on the surface chemistry of the microchannel, bulk fluid flow as a result of electroosmotic flow (EOF).

As will be appreciated by those in the art, the electrophoresis module cantake on a variety of forms, and generally comprises an electrophoretic microchannel and associated electrodes to apply an electric field to the electrophoretic microchannel. Waste fluid outlets and fluid reservoir chambers are present as required.

The electrodes comprise pairs of electrodes, either a single pair, or, as described in U.S. Pat. Nos. 5,126,022 and 5,750,015, a plurality of pairs. Single pairs generally have one electrode at each end of the electrophoretic pathway. Multiple electrode pairs may be used to precisely control the movement of sample components, such that the sample components may be continuously subjected to a plurality of electric fields either simultaneously or sequentially.

In a preferred embodiment, electrophoretic gel media may also be used. By varying the pore size of the media, employing two or more gel media of different porosity, and/or providing a pore size gradient, separation of sample components can be maximized. Gel media for separation based on size are known, and include, but are not limited to, polyacrylamide and agarose. One preferred electrophoretic separation matrix is described in U.S. Pat. No. 5,135,627, hereby incorporated by reference, that describes the use of “mosaic matrix”, formed by polymerizinga dispersion of microdomains (“dispersoids”) and a polymeric matrix. This allows enhanced separation of target analytes, particularly nucleic acids. Similarly, U.S. Pat. No. 5,569,364, hereby incorporated by reference, describes separation media for electrophoresis comprising submicron to above-micron sized cross-linked gel particles that find use in microfluidic systems. U.S. Pat. No. 5,631,337, hereby incorporated by reference, describes the use of thermoreversible hydrogels comprising polyacrylamide backbones with N-substituents that serve to provide hydrogen bonding groups for improved electrophoretic separation. See also U.S. Pat. Nos. 5,061,336 and 5,071,531, directed to methods of casting gels in capillary tubes, hereby incorporated by reference.

In a preferred embodiment, the devices of the invention include a reaction module. This can include either physical, chemical or biological alteration of one or more sample components. Alternatively, it may include a reaction module wherein the target analyte alters a second moiety that can then be detected; for example, if the target analyte is an enzyme, the reaction chamber may comprise an enzyme substrate that upon modification by the target analyte, can then be detected. In this embodiment, the reaction module may contain the necessary reagents, or they may be stored in a storage module and pumped as outlined herein to the reaction module as needed.

In a preferred embodiment, the reaction module includes a chamber for the chemical modification of all or part of the sample. For example, chemical cleavage of sample components (CNBr cleavage of proteins, etc.) or chemical cross-linking can be done. PCT US97/07880, hereby incorporated by reference, lists a large number of possible chemical reactions that can be done using the chambers and component devices of the invention, including amide formation, acylation, alkylation, reductive amination, Mitsunobu, Diels Alder and Mannich reactions, Suzuki and Stille coupling, chemical labeling, etc.

In a preferred embodiment, the reaction module includes a chamber for the biological alteration of all or part of the sample. For example, enzymatic processes including nucleic acid amplification, hydrolysis of sample components or the hydrolysis of substrates by an enzyme target analyte, the addition or removal of detectable labels, the addition or removal of phosphate groups, etc., as further described below.

The devices of the invention are used to detect target analytes. “Target analyte” and grammatical equivalents herein are used to refer to analytes to be detected or quantified. “Contamination analyte” and grammatical equivalents herein are used to refer to analytes present in a sample that are not to be detected. These “contamination analytes” frequently interfere with the efficient detection of “target analytes”. Target analytes preferably bind to a binding ligand, as is more fully described below.

Target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc. and environmental samples, such as, soil, water, air, pants, and the like; and manufactured products, etc.

As will be appreciated by those in the art, a large number of target analytes may be manipulated and subsequently detected using the present methods; basically, any target analyte for which a binding ligand, described herein, may be made may be detected using the methods of the invention.

Suitable target analytes include organic and inorganic molecules, including biomolecules. In a preferred embodiment, the target analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including prokaryotic (such as pathogenic bacteria) and eukaryotic cells, including mammalian tumor cells); viruses (including retroviruses, herpesviruses, adenoviruses, lentiviruses, etc.); and spores; etc. Particularly preferred target analytes are environmental pollutants; nucleic acids; proteins (including enzymes, antibodies, antigens, growth factors, cytokines, etc); therapeutic and abused drugs; cells; and viruses.

In a preferred embodiment, the target analyte is a nucleic acid, as described above.

In a preferred embodiment, the present invention provides methods of manipulating and detecting target nucleic acids. By “target nucleic acid” or “target sequence” or grammatical equivalents herein means a nucleic acid sequence on a single strand of nucleic acid. The target sequence may be a portion of a gene, a regulatory sequence, genomicDNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 base pairs, with fragments of roughly 500 base pairs being preferred in some embodiments. As will be appreciated by those in the art, the complementary target sequence may take many forms. For example, it may be contained within a larger nucleic acid sequence, e.g. all or part of a gene or mRNA, a restriction fragment of a plasmid or genomic DNA, among others.

As is outlined more fully below, probes (including primers) are made to hybridize to target sequences to determine the presence or absence of the target sequence in a sample. Generally speaking, this term will be understood by those skilled in the art.

The target sequence may also be comprised of different target domains, which may be adjacent or separate. For example, when ligation chain reaction (LCR) techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

In a preferred embodiment, the target analyte is a protein. As will be appreciated by those in the art, there are a large number of possible proteinaceous target analytes that may be detected using the present invention.

Suitable protein analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses (e.g. influenza A and B viruses), paramyxoviruses (e.g. respiratory syncytial virus, parainfluenza viruses, mumps virus, measles virus, canine distemper virus), astroviruses, adenoviruses, coronaviruses, reoviruses (e.g. rotaviruses), togaviruses (e.g. rubella virus), parvoviruses (e.g. erythroviruses), poxviruses (e.g. variola virus, vaccinia virus), hepatitis viruses (including A, B, C, D (deltaviruses), and E), herpesviruses (e.g. herpes simplex virus, varicella-zoster virus, cytomegalovirus, Epstein-Barr virus), caliciviruses (e.g. Norwalk viruses), arenaviruses, rhabdoviruses (e.g. rabies virus), retroviruses (including HIV, HTLV-I and -II), papillomaviruses, polyomaviruses, picornaviruses (e.g. enteroviruses (e.g. poliovirus, coxsackievirus), parechoviruses, cardioviruses, rhinoviruses, aphthoviruses (e.g. foot-and-mouth disease virus), and hepatoviruses), flaviviruses (e.g. West Nile virus, pestiviruses, hepaciviruses), bunyaviruses (e.g. hantaviruses), filoviruses (e.g. Ebola virus) and the like); bacteria (including a wide variety of pathogenic and non-pathogenic prokaryotes of interest including Bacillus, e.g. B. anthracis; Vibrio, e.g. V. cholerae; Escherichia, e.g. Enterotoxigenic E. coli, Shigella, e.g. S. dysenteriae; Salmonella, e.g. S. typhi; Mycobacterium, e.g. M. tuberculosis, M. leprae; Clostridium, e.g. C. botulinum, C. tetani, C. difficile, C. perfringens; Cornyebacterium, e.g. C. diphtheriae; Streptococcus, e.g. S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. enterocolitica, Y. pseudotuberculosis, Y. pestis, Pseudomonas, e.g. P. aeruginosa, P. putida; Chlamydia, e.g. C. trachomatis; Bordetella, B. pertussis; Treponema, e.g. T. palladium; fungi and yeast (e.g. C. neoformans) and the like, and parasites (e.g. protozoa (e.g. G. lamblia, E. histolytica) and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, thrombin, tissue plasminogen activator (tPA); pancreatic disease indicators including amylase, lipase, chymotrypsin and trypsin; liver function enzymes and proteins including cholinesterase, bilirubin, and alkaline phosphatase; aldolase, prostatic acid phosphatase, terminal deoxynucleotidyl transferase, and bacterial and viral enzymes such as reverse transcriptase and HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cortisol, estradiol, follicle stimulating hormone (FSH), thyroid-stimulating hormone (TSH), leutinzing hormone (LH), progesterone and testosterone; and (4) lipids such as cholesterol, triglycerides, steroids and the like.

In addition, any of the molecules for which antibodies may be detected may be detected directly as well; that is, detection of virus or bacterial cells, therapeutic and abused drugs, etc, may be done directly.

Suitable analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242).

In a preferred embodiment, the target analyte is a nucleic acid and the microfluidic system of the invention allows amplification of the target nucleic acid. Suitable amplification techniques include, both target amplification and probe amplification, including, but not limited to, polymerase chain reaction (PCR), ligase chain reaction (LCR), strand displacement amplification (SDA), self-sustained sequence replication (3SR), QB replicase amplification (QBR), repair chain reaction (RCR), cycling probe technology or reaction (CPT or CPR), and nucleic acid sequence based amplification (NASBA). In this embodiment, the reaction reagents generally comprise at least one enzyme (generally polymerase), primers, and nucleoside triphosphates as needed.

General techniques for nucleic acid amplification are discussed below. In most cases, double stranded target nucleic acids are denatured to render them single stranded so as to permit hybridization of the primers and other probes. A preferred embodiment utilizes a thermal step, generally by raising the temperature of the reaction to about 95° C., although pH changes and other techniques such as the use of extra probes or nucleic acid binding proteins may also be used. Thus, as more fully described above, the reaction chambers of the invention can include thermal modules.

A probe nucleic acid (also referred to herein as a primer nucleic acid) is then contacted to the target sequence to form a hybridization complex. By “primer nucleic acid” herein is meant a probe nucleic acid that will hybridize to some portion, i.e. a domain, of the target sequence. Probes of the present invention are designed to be complementary to a target sequence (either the target sequence of the sample or to other probe sequences, as is described below), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. See for example, Tibanyenda et al., Eur J. Biochem. 139:19 (1984), Ebel et al. Biochem. 31:12083 (1992), incorporated by reference. Thus, by “substantially complementary” herein is meant that the probes are sufficiently complementary to the target sequences to hybridize under normal reaction conditions.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al., all of which are hereby incorporated by reference. Stringent conditions are sequence-dependent and will be different in different circumstances. For example, it is well known in the art that longer sequences hybridize specifically at higher temperatures. Thus, the specificity and selectivity of the probe can be adjusted by choosing proper lengths for the targeting domains and appropriate hybridization conditions. For example, when the target nucleic acid is genomic DNA, e.g., mammalian genomic DNA, the selectivity of the targeting domains must be high enough to identify the correct base in 3×10⁹ in order to allow processing directly from genomic DNA. However, in situations in which a portion of the genomic DNA is isolated first from the rest of the DNA, e.g., by separating one or more chromosomes from the rest of the chromosomes, the selectivity or specificity of the probe is less important.

An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993), incorporated by reference. Generally, stringent conditions are selected to be about 5-10° C. lower than the thermal melting point (Tₘ) for the specific sequence at a defined ionic strength, pH. The Tₘ is the temperature (under defined ionic strength, pH and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. about 10 to 50 nucleotides) and at least about 60° C. for long probes (e.g. greater than about 50 nucleotides). Stringent conditions may also be achieved with the addition of destabilizing agents such as formamide. The hybridization conditions may also vary when a non-ionic backbone, e.g. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc. These parameters may also be used to control non-specific binding, as is generally outlined in U.S. Pat. No. 5,681,697. Thus, it may be desirable to perform certain steps at higher stringency conditions to reduce non-specific binding.

The size of the primer nucleic acid may vary, as will be appreciated by those in the art, in general varying from 5 to 500 nucleotides in length, with primers of between 10 and 100 being preferred, between 15 and 50 being particularly preferred, and from 10 to 35 being especially preferred, depending on the use and amplification technique.

In addition, the different amplification techniques may have further requirements of the primers, as is more fully described below.

Once the hybridization complex between the primer and the target sequence has been formed, an enzyme, sometimes termed an “amplification enzyme”, is used to modify the primer. As for all the methods outlined herein, the enzymes may be added at any point during the assay, either prior to, during, or after the addition of the primers. The identification of the enzyme will depend on the amplification technique used, as is more fully outlined below. Similarly, the modification will depend on the amplification technique, as outlined below, although generally the first step of all the reactions herein is an extension of the primer, that is, nucleotides are added to the primer to extend its length.

Once the enzyme has modified the primer to form a modified primer, the hybridization complex is disassociated. Generally, the amplification steps are repeated for a period of time to allow a number of cycles, depending on the number of copies of the original target sequence and the sensitivity of detection, with cycles ranging from 1 to thousands, with from 10 to 100 cycles being preferred and from 20 to 50 cycles being especially preferred.

After a suitable time or amplification, the modified primer can be moved to a detection module and detected.

In a preferred embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), and nucleic acid sequence based amplification (NASBA).

In a preferred embodiment, the target amplification technique is PCR. The polymerase chain reaction (PCR) is widely used and described, and involve the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”, “panhandle PCR”, and “PCR select cDNA subtraction”, among others. In one embodiment, the amplification technique is not PCR.

In general, PCR may be briefly described as follows. A double stranded target nucleic acid is denatured, generally by raising the temperature, and then cooled in the presence of an excess of a PCR primer, which then hybridizes to the first target strand. A DNA polymerase then acts to extend the primer, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denatureation, annealing and extension. The particulars of PCR are well known, and include the use of a thermostabile polymerase such as TaqI polymerase and thermal cycling.

Accordingly, the PCR reaction requires at least one PCR primer and a polymerase.

In a preferred embodiment, the target amplification technique is SDA. Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby expressly incorporated by reference in their entirety.

In general, SDA may be described as follows. A single stranded target nucleic acid, usually a DNA target sequence, is contacted with an SDA primer. An “SDA primer” generally has a length of 25-100 nucleotides, with SDA primers of approximately 35 nucleotides being preferred. An SDA primer is substantially complementary to a region at the 3′ end of the target sequence, and the primer has a sequence at its 5′ end (outside of the region that is complementary to the target) that is a recognition sequence for a restriction endonuclease, sometimes referred to herein as a “nicking enzyme” or a “nicking endonuclease”, as outlined below. The SDA primer then hybridizes to the target sequence. The SDA reaction mixture also contains a polymerase (an “SDA polymerase”, as outlined below) and a mixture of all four deoxynucleoside-triphosphates (also called deoxynucleotides or dNTPs, i.e. dATP, dTTP, dCTP and dGTP), at least one species of which is a substituted or modified dNTP; thus, the SDA primer is modified, i.e. extended, to form a modified primer, sometimes referred to herein as a “newly synthesized strand”. The substituted dNTP is modified such that it will inhibit cleavage in the strand containing the substituted dNTP but will not inhibit cleavage on the other strand. Examples of suitable substituted dNTPs include, but are not limited, 2′-deoxyadenosine 5′-O-(1-thiotriphosphate), 5-methyldeoxycytidine 5′-triphosphate, 2′-deoxyuridine 5′-triphosphate, adn 7-deaza-2′-deoxyguanosine 5′-triphosphate. In addition, the substitution of the dNTP may occur after incorporation into a newly synthesized strand; for example, a methylase may be used to add methyl groups to the synthesized strand. In addition, if all the nucleotides are substituted, the polymerase may have 5′-3′ exonuclease activity. However, if less than all the nucleotides are substituted, the polymerase preferably lacks 5′-3′ exonuclease activity.

As will be appreciated by those in the art, the recognition site/endonuclease pair can be any of a wide variety of known combinations. The endonuclease is chosen to cleave a strand either at the recognition site, or either 3′ or 5′ to it, without cleaving the complementary sequence, either because the enzyme only cleaves one strand or because of the incorporation of the substituted nucleotides. Suitable recognition site/endonuclease pairs are well known in the art; suitable endonucleases include, but are not limited to, HincII, HindII, Aval, Fnu4HI, TthIIII, NcII, BstXI, BamI, etc. A chart depicting suitable enzymes, and their corresponding recognition sites and the modified dNTP to use is found in U.S. Pat. No. 5,455,166, hereby expressly incorporated by reference.

Once nicked, a polymerase (an “SDA polymerase”) is used to extend the newly nicked strand, 5′-3′, thereby creating another newly synthesized strand. The polymerase chosen should be able to initiate 5′-3′ polymerization at a nick site, should also displace the polymerized strand downstream from the nick, and should lack 5′-3′ exonuclease activity (this may be additionally accomplished by the addition of a blocking agent). Thus, suitable polymerases in SDA include, but are not limited to, the Klenow fragment of DNA polymerase I, SEQUENASE 1.0 and SEQUENASE 2.0 (U.S. Biochemical), T5 DNA polymerase and Phi29 DNA polymerase.

Accordingly, the SDA reaction requires, in no particular order, an SDA primer, an SDA polymerase, a nicking endonuclease, and dNTPs, at least one species of which is modified.

In general, SDA does not require thermocycling. The temperature of the reaction is generally set to be high enough to prevent non-specific hybridization but low enough to allow specific hybridization; this is generally from about 37° C. to about 42° C., depending on the enzymes.

In a preferred embodiment, as for most of the amplification techniques described herein, a second amplification reaction can be done using the complementary target sequence, resulting in a substantial increase in amplification during a set period of time. That is, a second primer nucleic acid is hybridized to a second target sequence, that is substantially complementary to the first target sequence, to form a second hybridization complex. The addition of the enzyme, followed by disassociation of the second hybridization complex, results in the generation of a number of newly synthesized second strands.

In a preferred embodiment, the target amplification technique is nucleic acid sequence based amplification (NASBA). NASBA is generally described in U.S. Pat. No. 5,409,818; Sooknanan et al., Nucleic Acid Sequence-Based Amplification, Ch. 12 (pp. 261-285) of Molecular Methods for Virus Detection, Academic Press, 1995; and “Profiting from Gene-based Diagnostics”, CTB International Publishing Inc., N.J., 1996, all of which are incorporated by reference. NASBA is very similar to both TMA and QBR. Transcription mediated amplification (TMA) is generally described in U.S. Pat. Nos. 5,399,491; 5,888,779; 5,705,365; 5,710,029, all of which are incorporated by reference. The main difference between NASBA and TMA is that NASBA utilizes the addition of RNAse H to effect RNA degradation, and TMA relies on inherent RNAse H activity of the reverse transcriptase.

In general, these techniques may be described as follows. A single stranded target nucleic acid, usually an RNA target sequence (sometimes referred to herein as “the first target sequence” or “the first template”), is contacted with a first primer, generally referred to herein as a “NASBA primer” (although “TMA primer” is also suitable). Starting with a DNA target sequence is described below. These primers generally have a length of 25-100 nucleotides, with NASBA primers of approximately 50-75 nucleotides being preferred. The first primer is preferably a DNA primer that has at its 3′ end a sequence that is substantially complementary to the 3′ end of the first template. The first primer also has an RNA polymerase promoter at its 5′ end (or its complement (antisense), depending on the configuration of the system). The first primer is then hybridized to the first template to form a first hybridization complex. The reaction mixture also includes a reverse transcriptase enzyme (an “NASBA reverse transcriptase”) and a mixture of the four dNTPs, such that the first NASBA primer is modified, i.e. extended, to form a modified first primer, comprising a hybridization complex of RNA (the first template) and DNA (the newly synthesized strand).

By “reverse transcriptase” or “RNA-directed DNA polymerase” herein is meant an enzyme capable of synthesizing DNA from a DNA primer and an RNA template. Suitable RNA-directed DNA polymerases include, but are not limited to, avian myeloblastosis virus reverse transcriptase (“AMV RT”) and the Moloney murine leukemia virus RT. When the amplification reaction is TMA, the reverse transcriptase enzyme further comprises a RNA degrading activity as outlined below.

In addition to the components listed above, the NASBA reaction also includes an RNA degrading enzyme, also sometimes referred to herein as a ribonuclease, that will hydrolyze RNA of an RNA:DNA hybrid without hydrolyzing single- or double-stranded RNA or DNA. Suitable ribonucleases include, but are not limited to, RNase H from E. coli and calf thymus.

The ribonuclease activity degrades the first RNA template in the hybridization complex, resulting in a disassociation of the hybridization complex leaving a first single stranded newly synthesized DNA strand, sometimes referred to herein as “the second template”.

In addition, the NASBA reaction also includes a second NASBA primer, generally comprising DNA (although as for all the probes herein, including primers, nucleic acid analogs may also be used). This second NASBA primer has a sequence at its 3′ end that is substantially complementary to the 3′ end of the second template, and also contains an antisense sequence for a functional promoter and the antisense sequence of a transcription initiation site. Thus, this primer sequence, when used as a template for synthesis of the third DNA template, contains sufficient information to allow specific and efficient binding of an RNA polymerase and initiation of transcription at the desired site. Preferred embodiments utilizes the antisense promoter and transcription initiation site of the T7 RNA polymerase, although other RNA polymerase promoters and initiation sites can be used as well, as outlined below.

The second primer hybridizes to the second template, and a DNA polymerase, also termed a “DNA-directed DNA polymerase”, also present in the reaction, synthesizes a third template (a second newly synthesized DNA strand), resulting in second hybridization complex comprising two newly synthesized DNA strands.

Finally, the inclusion of an RNA polymerase and the required four ribonucleoside triphosphates (ribonucleotides or NTPs) results in the synthesis of an RNA strand (a third newly synthesized strand that is essentially the same as the first template). The RNA polymerase, sometimes referred to herein as a “DNA-directed RNA polymerase”, recognizes the promoter and specifically initiates RNA synthesis at the initiation site. In addition, the RNA polymerase preferably synthesizes several copies of RNA per DNA duplex. Preferred RNA polymerases include, but are not limited to, T7 RNA polymerase, and other bacteriophage RNA polymerases including those of phage T3, phage φII, Salmonella phage sp6, or Pseudomonas phage gh--1.

In some embodiments, TMA and NASBA are used with starting DNA target sequences. In this embodiment, it is necessary to utilize the first primer comprising the RNA polymerase promoter and a DNA polymerase enzyme to generate a double stranded DNA hybrid with the newly synthesized strand comprising the promoter sequence. The hybrid is then denatured and the second primer added.

Accordingly, the NASBA reaction requires, in no particular order, a first NASBA primer, a second NASBA primer comprising an antisense sequence of an RNA polymerase promoter, an RNA polymerase that recognizes the promoter, a reverse transcriptase, a DNA polymerase, an RNA degrading enzyme, NTPs and dNTPs, in addition to the detection components outlined below.

These components result in a single starting RNA template generating a single DNA duplex; however, since this DNA duplex results in the creation of multiple RNA strands, which can then be used to initiate the reaction again, amplification proceeds rapidly.

Accordingly, the TMA reaction requires, in no particular order, a first TMA primer, a second TMA primer comprising an antisense sequence of an RNA polymerase promoter, an RNA polymerase that recognizes the promoter, a reverse transcriptase with RNA degrading activity, a DNA polymerase, NTPs and dNTPs, in addition to the detection components outlined below.

These components result in a single starting RNA template generating a single DNA duplex; however, since this DNA duplex results in the creation of multiple RNA strands, which can then be used to initiate the reaction again, amplification proceeds rapidly.

In a preferred embodiment, the amplification technique is signal amplification. Signal amplification involves the use of limited number of target molecules as templates to either generate multiple signalling probes or allow the use of multiple signalling probes. Signal amplification strategies include LCR, CPT, Invader™, and the use of amplification probes in sandwich assays.

In a preferred embodiment, the signal amplification technique is the oligonucleotide ligation assay (OLA), sometimes referred to as the ligation chain reaction (LCR). The method can be run in two different ways; in a first embodiment, only one strand of a target sequence is used as a template for ligation (OLA); alternatively, both strands may be used (OLA). See generally U.S. Pat. Nos. 5,185,243 and 5,573,907; EP 0 320 308 B1; EP 0 336 731 B1; EP 0 439 182 B1; WO 90/01069; WO 89/12696; and WO 89/09835; and U.S. patent application Ser. Nos. 60/078,102 and 60/073,011, all of which are incorporated by reference.

In a preferred embodiment, the single-stranded target sequence comprises a first target domain and a second target domain, and a first LCR primer and a second LCR primer nucleic acids are added, that are substantially complementary to its respective target domain and thus will hybridize to the target domains. These target domains may be directly adjacent, i.e. contiguous, or separated by a number of nucleotides, i.e., a “gap”. If they are non-contiguous, nucleotides are added along with means to join nucleotides, such as a polymerase, that will add the nucleotides to one of the primers. The two LCR primers are then covalently attached, for example using a ligase enzyme such as is known in the art. This forms a first hybridization complex comprising the ligated probe and the target sequence. This hybridization complex is then denatured (disassociated), and the process is repeated to generate a pool of ligated probes.

In a preferred embodiment, LCR is done for two strands of a double-stranded target sequence. The target sequence is denatured, and two sets of probes are added: one set as outlined above for one strand of the target, and a separate set (i.e. third and fourth primer probe nucleic acids) for the other strand of the target. In a preferred embodiment, the first and third probes will hybridize, and the second and fourth probes will hybridize, such that amplification can occur. That is, when the first and second probes have been attached, the ligated probe can now be used as a template, in addition to the second target sequence, for the attachment of the third and fourth probes. Similarly, the ligated third and fourth probes will serve as a template for the attachment of the first and second probes, in addition to the first target strand. In this way, an exponential, rather than just a linear, amplification can occur. A variation of LCR utilizes a “chemical ligation” of sorts, as is generally outlined in U.S. Pat. Nos. 5,616,464 and 5,767,259, both of which are hereby expressly incorporated by reference in their entirety. In this embodiment, similar to LCR, a pair of primers are utilized, wherein the first primer is substantially complementary to a first domain of the target and the second primer is substantially complementary to an adjacent second domain of the target (although, as for LCR, if a “gap” exists, a polymerase and dNTPs may be added to “fill in” the gap). Each primer has a portion that acts as a “side chain” that does not bind the target sequence and acts one half of a stem structure that interacts non-covalently through hydrogen bonding, salt bridges, van der Waal's forces, etc. Preferred embodiments utilize substantially complementary nucleic acids as the side chains. Thus, upon hybridization of the primers to the target sequence, the side chains of the primers are brought into spatial proximity, and, if the side chains comprise nucleic acids as well, can also form side chain hybridization complexes.

At least one of the side chains of the primers comprises an activatable cross-linking agent, generally covalently attached to the side chain, that upon activation, results in a chemical cross-link or chemical ligation. The activatible group may comprise any moiety that will allow cross-linking of the side chains, and include groups activated chemically, photonically and thermally, with photoactivatable groups being preferred. In some embodiments a single activatable group on one of the side chains is enough to result in cross-linking via interaction to a functional group on the other side chain; in alternate embodiments, activatable groups are required on each side chain.

Once the hybridization complex is formed, and the cross-linking agent has been activated such that the primers have been covalently attached, the reaction is subjected to conditions to allow for the disassociation of the hybridization complex, thus freeing up the target to serve as a template for the next ligation or cross-linking. In this way, signal amplification occurs, and can be detected as outlined herein.

In a preferred embodiment the signal amplification technique is RCA. Rolling-circle amplification is generally described in Baner et al. (1998) Nuc. Acids Res. 26:5073-5078; Barany, F. (1991) Proc. Natl. Acad. Sci. USA 88:189-193; Lizardi et al. (1998) Nat. Genet. 19:225-232; Zhang et al. Gene 211:277 (1998); and Daubendiek et al., Nature Biotech. 15:273 (1997); all of which are incorporated by reference in their entirety.

In general, RCA may be described as follows. First, as is outlined in more detail below, a single RCA probe is hybridized with a target nucleic acid. Each terminus of the probe hybridizes adjacently on the target nucleic acid (or alternatively, there are intervening nucleotides that can be “filled in” using a polymerase and dNTPs, as outlined below) and the OLA assay as described above occurs. When ligated, the probe is circularized while hybridized to the target nucleic acid. Addition of a primer, a polymerase and dNTPs results in extension of the circular probe. However, since the probe has no terminus, the polymerase continues to extend the probe repeatedly. Thus, this results in amplification of the circular probe. This very large concatamer can be detected intact, as described below, or can be cleaved in a variety of ways to form smaller amplicons for detection as outlined herein.

Accordingly, in an preferred embodiment, a single oligonucleotide is used both for OLA and as the circular template for RCA (referred to herein as a “padlock probe” or a “RCA probe”). That is, each terminus of the oligonucleotide contains sequence complementary to the target nucleic acid and functions as an OLA primer as described above. That is, the first end of the RCA probe is substantially complementary to a first target domain, and the second end of the RCA probe is substantially complementary to a second target domain, adjacent (either directly or indirectly, as outlined herein) to the first domain. Hybridization of the probe to the target nucleic acid results in the formation of a hybridization complex. Ligation of the “primers” (which are the discrete ends of a single oligonucleotide, the RCA probe) results in the formation of a modified hybridization complex containing a circular probe i.e. an RCA template complex. That is, the oligonucleotide is circularized while still hybridized with the target nucleic acid. This serves as a circular template for RCA. Addition of a primer, a polymerase and the required dNTPs to the RCA template complex results in the formation of an amplified product nucleic acid. Following RCA, the amplified product nucleic acid is detected as outlined herein. This can be accomplished in a variety of ways; for example, the polymerase may incorporate labeled nucleotides; a labeled primer may be used, or alternatively,a label probe is used that is substantially complementary to a portion of the RCA probe and comprises at least one label is used.

Accordingly, the present invention provides RCA probes (sometimes referred to herein as “rolling circle probes (RCPs) or “padlock probes” (PPs)). The RCPs may comprise any number of elements, including a first and second ligation sequence, a cleavage site, a priming site, a capture sequence, nucleotide analogs, and a label sequence.

In a preferred embodiment, the RCP comprises first and second ligation sequences. As outlined above for OLA, the ligation sequences are substantially complementary to adjacent domains of the target sequence. The domains may be directly adjacent (i.e. with no intervening bases between the 3′ end of the first and the 5′ of the second) or indirectly adjacent, with from 1 to 100 or more bases in between.

In a preferred embodiment, the RCPs comprise a cleavage site, such that either after or during the rolling circle amplification, the RCP concatamer may be cleaved into amplicons. In some embodiments, this facilitates the detection, since the amplicons are generally smaller and exhibit favorable hybridization kinetics on a surface. As will be appreciated by those in the art, the cleavage site can take on a number of forms, including, but not limited to, the use of restriction sites in the probe, the use of ribozyme sequences, or through the use or incorporation of nucleic acid cleavage moieties.

In a preferred embodiment, the padlock probe contains a restriction site. The restriction endonuclease site allows for cleavage of the long concatamers that are typically the result of RCA into smaller individual units that hybridize either more efficiently or faster to surface bound capture probes. Thus, following RCA (or in some cases, during the reaction), the product nucleic acid is contacted with the appropriate restriction endonuclease. This results in cleavage of the product nucleic acid into smaller fragments. The fragments are then hybridized with the capture probe that is immobilized resulting in a concentration of product fragments onto the capture probe array. Again, as outlined herein, these fragments can be detected in one of two ways: either labelled nucleotides are incorporated during the replication step, for example either as labeled individual dNTPs or through the use of a labeled primer, or an additional label probe is added.

In a preferred embodiment, the restriction site is a single-stranded restriction site chosen such that its complement occurs only once in the RCP.

In a preferred embodiment, the cleavage site is a ribozyme cleavage site as is generally described in Daubendiek et al., Nature Biotech. 15:273 (1997), hereby expressly incorporated by reference. In this embodiment, by using RCPs that encode catalytic RNAs, NTPs and an RNA polymerase, the resulting concatamer can self cleave, ultimately forming monomeric amplicons.

In a preferred embodiment, cleavage is accomplished using DNA cleavage reagents. For example, as is known in the art, there are a number of intercalating moieties that can effect cleavage, for example using light.

In a preferred embodiment, the RCPs do not comprise a cleavage site. Instead, the size of the RCP is designed such that it may hybridize “smoothly” to many capture probes on a surface. Alternatively, the reaction may be cycled such that very long concatamers are not formed.

In a preferred embodiment, the RCPs comprise a priming site, to allow the binding of a DNA polymerase primer. As is known in the art, many DNA polymerases-require double stranded nucleic acid and a free terminus to allow nucleic acid synthesis. However, in some cases, for example when RNA polymerases are used, a primer may not be required (see Daubendiek, supra). Similarly, depending on the size and orientation of the target strand, it is possible that a free end of the target sequence can serve as the primer; see Baner et al, supra.

Thus, in a preferred embodiment, the padlock probe also contains a priming site for priming the RCA reaction. That is, each padlock probe comprises a sequence to which a primer nucleic acid hybridizes forming a template for the polymerase. The primer can be found in any portion of the circular probe. In a preferred embodiment, the primer is located at a discrete site in the probe. In this embodiment, the primer site in each distinct padlock probe is identical, although this is not required. Advantages of using primer sites with identical sequences include the ability to use only a single primer oligonucleotide to prime the RCA assay with a plurality of different hybridization complexes. That is, the padlock probe hybridizes uniquely to the target nucleic acid to which it is designed. A single primer hybridizes to all of the unique hybridization complexes forming a priming site for the polymerase. RCA then proceeds from an identical locus within each unique padlock probe of the hybridization complexes.

In an alternative embodiment, the primer site can overlap, encompass, or reside within any of the above-described elements of the padlock probe. That is, the primer can be found, for example, overlapping or within the restriction site or the identifier sequence. In this embodiment, it is necessary that the primer nucleic acid is designed to base pair with the chosen primer site.

In a preferred embodiment, the RCPs comprise a capture sequence. A capture sequence, as is outlined herein, is substantially complementary to a capture probe, as outlined herein.

In a preferred embodiment, the RCPs comprise a label sequence; i.e. a sequence that can be used to bind label probes and is substantially complementary to a label probe. In one embodiment, it is possible to use the same label sequence and label probe for all padlock probes on an array; alternatively, each padlock probe can have a different label sequence.

In a preferred embodiment, the RCP/primer sets are designed to allow an additional level of amplification, sometimes referred to as “hyperbranching” or “cascade amplification”. As described in Zhang et al., supra, by using several priming sequences and primers, a first concatamer can serve as the template for additional concatamers. In this embodiment, a polymerase that has high displacement activity is preferably used. In this embodiment, a first antisense primer is used, followed by the use of sense primers, to generate large numbers of concatamers and amplicons, when cleavage is used.

Thus, the invention provides for methods of detecting using RCPs as described herein. Once the ligation sequences of the RCP have hybridized to the target, forming a first hybridization complex, the ends of the RCP are ligated together as outlined above for OLA. The RCP primer is added, if necessary, along with a polymerase and dNTPs (or NTPs, if necessary).

The polymerase can be any polymerase as outlined herein, but is preferably one lacking 3′ exonuclease activity (3′ exo⁻). Examples of suitable polymerase include but are not limited to exonuclease minus DNA Polymerase I large (Klenow) Fragment, Phi29 DNA polymerase, Taq DNA Polymerase and the like. In addition, in some embodiments, a polymerase that will replicate single-stranded DNA (i.e. without a primer forming a double stranded section) can be used.

Thus, in a preferred embodiment the OLA/RCA is performed in solution followed by restriction endonuclease cleavage of the RCA product. The cleaved product is then applied to an array as described herein. The incorporation of an endonuclease site allows the generation of short, easily hybridizable sequences. Furthermore, the unique capture sequence in each rolling circle padlock probe sequence allows diverse sets of nucleic acid sequences to be analyzed in parallel on an array, since each sequence is resolved on the basis of hybridization specificity.

In a preferred embodiment, the polymerase creates more than 100 copies of the circular DNA. In more preferred embodiments the polymerase creates more than 1000 copies of the circular DNA; while in a most preferred embodiment the polymerase creates more than 10,000 copies or more than 50,000 copies of the template.

The RCA as described herein finds use in allowing highly specific and highly sensitive detection of nucleic acid target sequences. In particular, the method finds use in improving the multiplexing ability of DNA arrays and eliminating costly sample or target preparation. As an example, a substantial savings in costcan be realized by directly analyzing genomic DNA on an array, rather than employing an intermediate PCR amplification step. The method finds use in examining genomic DNA and other samples: including mRNA.

In addition the RCA finds use in allowing rolling circle amplification products to be easily detected by hybridization to probes in a solid-phase format. An additional advantage of the RCA is that it provides the capability of multiplex analysis so that large numbers of sequences can be analyzed in parallel. By combining the sensitivity of RCA and parallel detection on arrays, many sequences can be analyzed directly from genomic DNA.

In a preferred embodiment, the signal amplification technique is CPT. CPT technology is described in a number of patents and patent applications, including U.S. Pat. Nos. 5,011,769; 5,403,711; 5,660,988; and 4,876,187, and PCT published applications WO95/05480, WO95/1416, and WO95/00667, and U.S. patent application Ser. No. 09/014,304, all of which are expressly incorporated by reference-in their entirety.

Generally, CPT may be described as follows. A CPT primer (also sometimes referred to herein as a “scissile primer”), comprises two probe sequences separated by a scissile linkage. The CPT primer is substantially complementary to the target sequence and thus will hybridize to it to form a hybridization complex. The scissile linkage is cleaved, without cleaving the target sequence, resulting in the two probe sequences being separated. The two probe sequences can thus be more easily disassociated from the target, and the reaction can be repeated any number of times. The cleaved primer is then detected as outlined herein.

By “scissile linkage” herein is meant a linkage within the scissile probe that can be cleaved when the probe is part of a hybridization complex, that is, when a double-stranded complex is formed. It is important that the scissile linkage cleave only the scissile probe and not the sequence to which it is hybridized (i.e. either the target sequence or a probe sequence), such that the target sequence may be reused in the reaction for amplification of the signal. As used herein, the scissile linkage, is any connecting chemical structure which joins two probe sequences and which is capable of being selectively cleaved without cleavage of either the probe sequences or the sequence to which the scissile probe is hybridized. The scissile linkage may be a single bond, or a multiple unit sequence. As will be appreciated by those in the art, a number of possible scissile linkages may be used.

In a preferred embodiment, the scissile linkage comprises RNA. This system, as outline aove, is based on the fact that certain double-stranded nucleases, particularly ribonucleases, will nick or excise RNA nucleosides from a RNA:DNA hybridization complex. Of particular use in this embodiment is RNAse H, Exo III, and reverse transcriptase.

In one embodiment, the entire scissile probe is made of RNA, the nicking is facilitated especially when carried out with a double-stranded ribonuclease, such as RNAse H or Exo III. RNA probes made entirely of RNA sequences are particularly useful because first, they can be more easily produced enzymatically, and second, they have more cleavage sites which are accessible to nicking or cleaving by a nicking agent, such as the ribonucleases. Thus, scissile probes made entirely of RNA do not rely on a scissile linkage since the scissile linkage is inherent in the probe.

In a preferred embodiment, Invader™ technology is used. Invader™ technology is based on structure-specific polymerases that cleave nucleic acids in a site-specific manner. Two probes are used: an “invader” probe and a “signaling” probe, that adjacently hybridize to a target sequence with a non-complementary overlap. The enzyme cleaves at the overlap due to its recognition of the “tail”, and releases the “tail”. This can then be detected. The Invader™ technology is described in U.S. Pat. Nos. 5,846,717; 5,614,402; 5,719,028; 5,541,311; and 5,843,669, all of which are hereby incorporated by reference.

Accordingly, the invention provides a first primer, sometimes referred to herein as an “invader primer”, that hybridizes to a first domain of a target sequence, and a second primer, sometimes referred to herein as the signaling primer, that hybridizes to a second domain of the target sequence. The first and second target domains are adjacent. The signaling primer further comprises an overlap sequence, comprising at least one nucleotide, that is perfectly complementary to at least one nucleotide of the first target domain, and a non-complementary “tail” region. The cleavage enzyme recognizes the overlap structure and the noncomplementary tail, and cleaves the tail from the second primer. Suitable cleavage enzymes are described in the Patents outlined above, and include, but are not limited to, 5′ thermostable nucleases from Thermus species, including Thermus aquaticus, Thermus flavus and Thermus thermophilus. The entire reaction is done isothermally at a temperature such that upon cleavage, the invader probe and the cleaved signaling probe come off the target strand, and new primers can bind. In this way large amounts of cleaved signaling probe (i.e. “tails”) are made. The uncleaved signaling probes are removed (for example by binding to a solid support such as a bead, either on the basis of the sequence or through the use of a binding ligand attached to the portion of the signaling probe that hybridizes to the target). The cleaved signalling probes are then detected as outlined herein.

In this way, a number of target molecules (sometimes referred to herein as “amplicons”) are made. One of skill in the art will recognize that subsequent analysis and detection of the amplification products may be done in a variety of ways. As is more fully outlined below, these reactions (that is, the products of these reactions) can be detected as generally outlined in U.S. patent application Ser. Nos. 09/458,553; 09/458,501; 09/572,187; 09/495,992; 09/344,217; 09/439,889; 09/438,209; 09/344,620; 09/478,727 and WO00/31148; PCTUS00/17422, all of which are expressly incorporated by reference in their entirety. In a preferred embodiment, target molecules are detected using a microfluidic system as described herein.

Detection labels such as radioactive isotopes, fluorescent molecules, phosphorescent molecules, enzymes, antibodies, ligands, etc. may also be incorporated directly into the amplification products, or alternatively can be coupled to detection molecules for subsequent detection and analysis. Preferred methods include chemiluminescence, using both Horseradish Peroxidase and/or Alkaline Phosphatase with substrates that produce photons as breakdown products (kits available from Amersham, Boehringer-Mannheim, and Life Technologies/Gibco BRL); color production using both Horseradish Peroxidase and/or Alkaline Phosphatase with substrates that produce a colored precipitate (kits available from Life Technologies/Gibco BRL, and Boehringer-Mannheim); chemifluorescence using Alkaline Phosphatase and the substrate AttoPhosJ Amersham or other substrates that produce fluorescent products; fluorescence using Cy-5 (Amersham), fluorescein, Alexa dyes (Molecular Dynamics) and other fluorescent tags; radioactivity using end-labeling, nick translation, random priming, or PCR to incorporate radioactive molecules into the ligation oligonucleotide or amplification product. Other methods for labeling and detection will be readily apparent to one skilled in the art.

In one embodiment, the detection labels are incorporated directly into the amplification products during amplification. Examples of detection labels that can be incorporated into amplified DNA or RNA include nucleotide analogs such as BrdUrd (Hoy and Schimke, Mutation Research 290:217-230 (1993)), BrUTP (Wasnick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res. 22:3226-3232 (1994)). A preferred nucleotide analog detection label for DNA is BrdUrd (BUDR triphosphate, Sigma), and a preferred nucleotide analog detection label for RNA is Biotin-16-uridine-5′ triphosphate (Biotin-16-dUTP, Boehringher Mannheim). Molecules that combine two or more of these detection labels are also contemplated for use in the disclosed methods.

Detection labels that are incorporated into amplified nucleic acid, such as biotin, can be subsequently detected using sensitive methods well-known in the art. For example, biotin can be detected using streptavidin-alkaline phosphatase conjugate (Tropix, Ind.), which is bound to the biotin and subsequently detected by chemiluminescence of suitable substrates (for example, chemiluminescence substrate CSPD; disodium, 3-(4-methoxyspiro-[1,2-dioxetane-3-2′ (5′ -chloro)tricyclo [3.3.1.1^{3,7-}] decane]-4-yl) phenyl phosphate; Tropix, Inc.). A preferred detection label for use in detection of amplified RNA is acridinium-ester-labeled DNA probe (GenProbe, Inc., as described by Arnold et al., Clinical Chemistry 35:1588-1594 (1989)). An acridinium-ester-labeled detection probe permits the detection of amplified RNA without washing because unhybridized probe can be destroyed with alkali (Arnold et al. (1989)).

Another embodiment utilizes a probe or primer labeled with any composition detectable by spectroscopic, photochemical, biochemical, immunochemical, electrical, optical or chemical means. Preferred labels in the present invention include spectral labels such as fluorescent dyes (e.g., fluorescein isothiocyanate, Texas red, rhodamine, dixogenin, biotin, and the like), radiolabels (e.g., ³H, ¹²⁵I, ³⁵S, ¹⁴C, ³²P, ³³P, etc.), enzymes (e.g., horse-radish peroxidase, alkaline phosphatase, etc.), spectral calorimetric labels such as colloidal gold or colored glass or plastic (e.g. polystyrene, polypropylene, latex, etc.) beads. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. Thus, a wide variety of labels may be used, with the choice of label depending on sensitivity required, ease of conjugation with the compound, stability requirements, available instrumentation, and disposal provisions.

The label may be coupled directly or indirectly to the molecule to be detected according to methods well known in the art. Non-radioactive labels are often attached by indirect means. Generally, a ligand molecule (e.g., biotin) is covalently bound to a nucleic acid such as a probe, primer, amplicon, YAC, BAC or the like. The ligand then binds to an anti-ligand (e.g, streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody. Labels can also be conjugated directly to signal generating compounds, e.g., by conjugation with an enzyme or fluorophore or chromophore.

Means of detecting labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available. In general, a detector which monitors a probe-target nucleic acid hybridization is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising a nucleic acid array with particular set of probes bound to the array is digitized for subsequent computer analysis.

Fluorescent labels are preferred labels, having the advantage of requiring fewer precautions in handling, and being amendable to high-throughput visualization techniques. Preferred labels are typically characterized by one or more of the following: high sensitivity, high stability, low background, low environmental sensitivity and high specificity in labeling. Fluorescent moieties, which are incorporated into the labels of the invention, are generally are known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes and flavin. Individual fluorescent compounds which have functionalities for linking to an element desirably detected in an apparatus or assay of the invention, or which can be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′ (vinylene-p-phenylene)bisbenzoxazole; p-bis(2- -methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone. Many fluorescent tags are commercially available from SIGMA chemical company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill in the art.

In a preferred embodiment, the amplification products obtained following the methods of the present invention are detected using conventional sequence-specific probe technology, such as the cross-linkable capture and reported probes described in U.S. Pat. Nos. 6,277,570; 6,005,093 and 6,187,532, the disclosures of which are incorporated by reference herein.

In another preferred embodiment, molecular beacons are employed as described in Leone et al., Nuc. Acids Res. 26:2150-55 (1995); Tyagi et al., Nature Biotech. 14:303-308 (1996); Kostritis et al., Science 279:1228-29 (1998); Tyagi et al. Nature Biotech. 16:49-53 (1998); Vet et al. Proc. Nat. Acad. Sci. USA 96:6394-99 (1999) and Marras et al., Genet. Anal. Biomol. Eng. 14:151-156 (1999), all of which are incorporated by reference. Briefly, molecular beacons are dual-labeled oligonucleotides having a fluorescent reporter group at one end and a fluorescent quencher group at the other end, which in the absence of target form an internal hairpin that brings the reported and quencher in physical proximity so as to quench the fluorescent signal. In the presence of target, the probe molecule unfolds and hybridizes to the target, resulting in separation of the reporter and quencher and emission of a fluorescent signal upon stimulation. In preferred embodiments, the quencher comprises Dabcyl (4-(4′-dimethylaminophenylazo)benzoic acid) and the fluorophore comprises fluorescein, tetrachloro-6-carboxyfluorescein, hetra-6-carboxyfluorescein, tetramethylrhodamine or rhodamine-X. Alternatively, detection techniques such as fluorescence resonance energy transfer (FRET) (Ota et al., Nuc. Acids. Res. 26:735-43 (1998)) and TaqManJ (Livak et al., PCR Methods Appl. 4:357-62 (1995); Livak, Genet. Anal. 14:143-49 (1999); Chen et al., J. Med. Virol. 65:250-56(2001)), all of which are incorporated by reference, can be employed.

In an alternative embodiment, the circular targets are detected on a micro-formatted multiplex or matrix devices (e.g., DNA chips) (see M. Barinaga, 253 Science, pp. 1489, 1991; W. Bains, 10 Bio/Technology, pp. 757-758, 1992). These methods usually attach specific DNA sequences to very small specific areas of a solid support, such as micro-wells of a DNA chip. In one variant, the invention is adapted to solid phase arrays for the rapid and specific detection of multiple polymorphic nucleotides, e.g., SNPs. Typically, an oligonucleotide such as the ligation oligonucleotide of the present invention is linked to a solid support and a target nucleic acid is hybridized to the oligonucleotide. Either the oligonucleotide, or the target, or both, can be labeled, typically with a fluorophore. Where the target is labeled, hybridization is detected by detecting bound fluorescence. Where the oligonucleotide is labeled, hybridization is typically detected by quenching of the label. Where both the oligonucleotide and the target are labeled, detection of hybridization is typically performed by monitoring a color shift resulting from proximity of the two bound labels. A variety of labeling strategies, labels, and the like, particularly for fluorescent based applications are described, supra.

In an alternative embodiment, unlabelled target nucleic acid or unlabelled amplification product of the target nucleic acid is detected. In one embodiment, the target nucleic acid sequence is comprised of different target domains, which may be adjacent or separate. The target nucleic acid is detected by hybridizing a first target domain to a capture probe in an array format. This first assay complex is detected by the addition of a second probe comprising a label or “label probe” which hybridizes to a second target domain, thereby forming a second assay complex. The “label probe” may comprise one or more labels as described above. Alternatively, once the capture and label probe are hybridized to the target nucleic acid, the capture and label probe are ligated together either chemically (e.g. photocrosslinked) or by a ligase. As known in the art, prior to ligation any gap between the capture and label probe is filled-in by, for example, either a polymerase that adds the nucleotides to at least one of the primers enzymatically as described above or by the hybridization of one or more additional probes to the gap region as needed.

In an alternative embodiment for detecting unlabelled target nucleic acid or its unlabelled amplification product, the target domain is hybridized to a capture probe in an array format to form an assay complex having at least one 5′ overhang and 3′ recessed end which serves as a substrate for a polymerase. Therefore, in one embodiment the overhang is filled-in by a polymerase that adds at least one labeled nucleotide to the overhang region. In optional embodiments, either the target nucleic acid or the capture probe is extended by the polymerase. In a preferred embodiment, the capture probe is extended by at least one labelled nucleotide.

The following examples serve to more fully describe the manner of using the above-described invention, as well as to set forth the best modes contemplated for carrying out various aspects of the invention. It is understood that these embodiments in no way serve to limit the scope of this invention. All references cited herein are expressly incorporated by reference in their entirety and for all purposes.

### EXAMPLES

### Example 1

Effects of Shaking, Membrane Flexibility, and Chamber Volume on Mixing

Microfluidic chambers having a flexible membrane were constructed by hand. The chambers were filled with buffer (50% formamide and 6×SSPE). With the exception of the control, the microfluidic chambers were placed on a rotary table shaker (Innova 4080) manufactured by New Brunswick Scientific at 300 rpm. Varying volumes of a Cy3 labeled 25-mer oligonucleotide solution was injected into the inlet port of all the microfluidic chambers. Mixing was monitored in real time by scanning, and monitoring the formation of fluorescent plumes in each chamber over time using methods well known to the skilled artisan: The effects of shaking, sample volume and shape of the flexible membrane on mixing were examined. The data are plotted as the percent fluorescent area versus time.

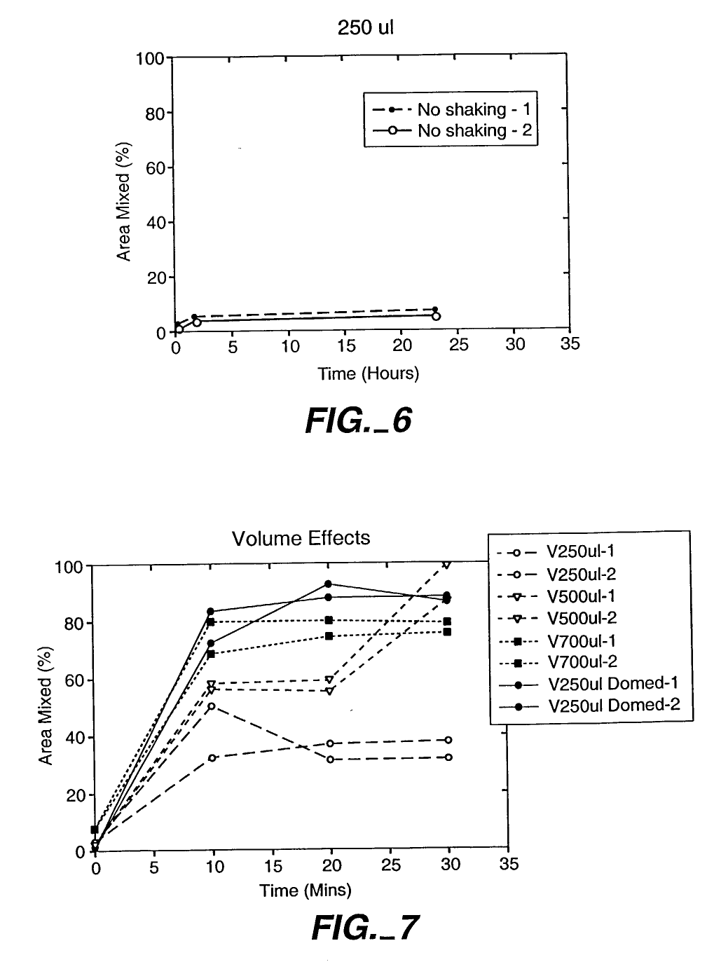

As shown in FIG. 6, the stationary (not shaken) 250 μl chambers having flexible membranes achieved only 7% mixing in more than 23 hours. In contrast, comparable volume chambers with a flexible membrane achieved 80% mixing within 10-25 minutes with rotary shaking (FIG. 7). The data also demonstrate that mixing efficiency is roughly proportional to the volume of the chamber, and that a domed flexible membrane significantly increases mixing efficiency (FIG. 7).

### Example 2

Effects of Shaking, Membrane Flexibility, and Chamber Volume on Hybridization Efficiency

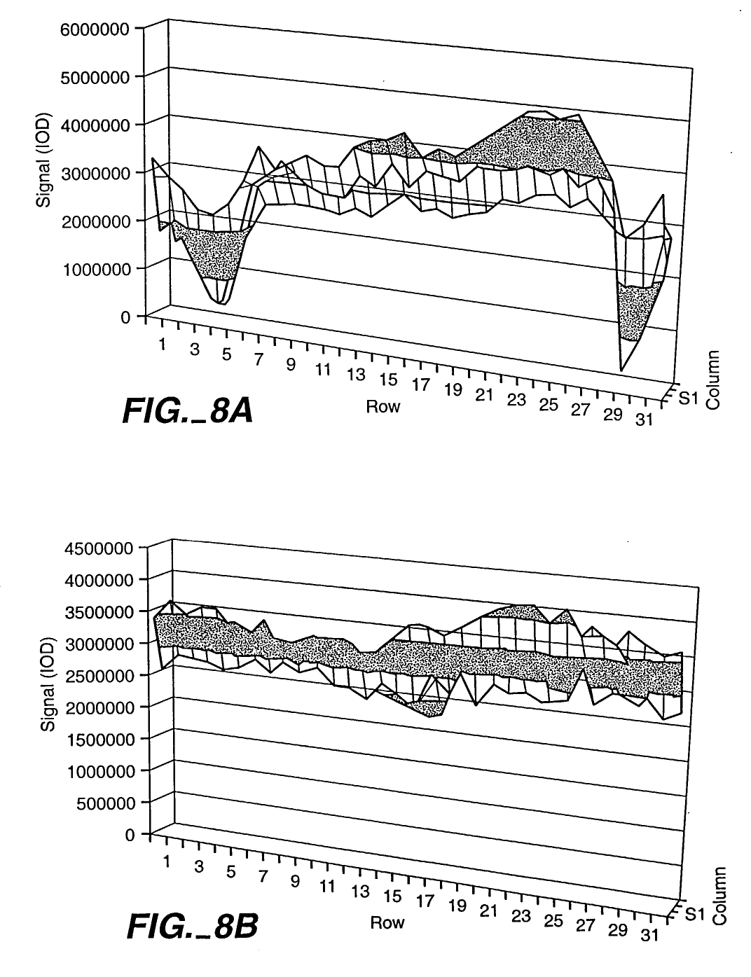

Microfluidic chambers having a flexible membrane were constructed by hand, as in Example 1. For this example the substrate had a pair of 25-mer-oligonucleotides in an array of 32 rows by 3 columns attached thereto. The chambers were filled with buffer (50% formamide and 6×SSPE). A microvolume of a solution with two Cy3 labeled 25-mers, complementary to those arrayed on the substrate, was injected into the inlet port of all the microfluidic chambers. One of the microfluidic chambers was placed on a rotary table shaker (Innova 4080 manufactured by New Brunswick Scientific) at 200 rpm and allowed to hybridize under appropriate conditions. The second was allowed to hybridize without shaking as a control. After 18 hours incubation the substrates were subjected to 3×water washes, dried and scanned at 400PMT. Referring to FIGS. 8A and 8B, the chamber with a flexible membrane subjected to shaking showed significantly more even distribution of hybridization over the entire array, indicating superior mixing and reagent exchange within the chamber.

### Example 3

Efficiency of Agitation

Generally, the hybridization process is a diffusion-limited process, which is extremely slow. The characteristic time, τ˜L²/D, where L is length and D is the diffusion coefficient, is typically about 17 hours with D˜1 μm²/s (20-mers) and a normal distance of 250 μm. The time will be much longer if a transverse distance which is in the order of ten thousands of μm is considered. Thus, in order to decrease the hybridization time, diffusion enhancement (e.g. a force) is required.

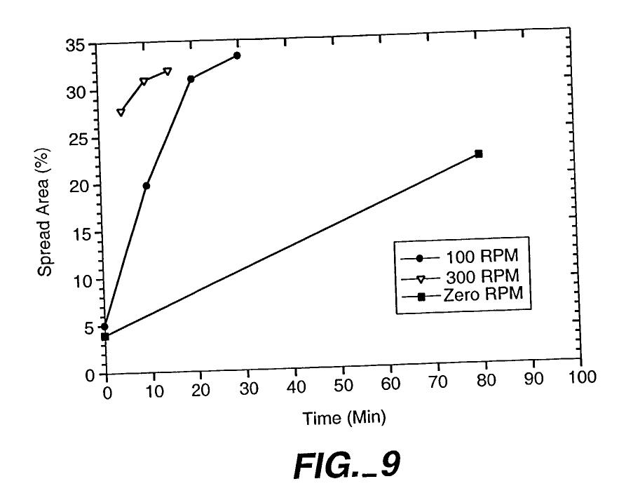

FIG. 9 shows the efficiency of agitation of a target nucleic acid in a microfluidic chamber comprising a microarray in which the radius of rotation was randomized from 0.25 to 1.0 inches at the indicated revolutions per minute (rpm). As the revolutions per minute increased from 0 to 300, the spread area increased substantially at each of the time points measured.

These results suggest that spatial homogeneity of the fluid sample is maintained if the local target replenishment rate (a flow variable) is higher than the local target consumption rate (reaction variable). Thus, the local target replenishment rate is related to the rotational rate, ω, which is related to the force exerted on the fluid (F(t)=ρω²r(t), where F is force per volume, ρ is the fluid density, r is the radius of rotation and t is time) and the variation of r causes a variation of force to mix the fluid.

The foregoing description, for purposes of explanation, used specific nomenclature to provide a thorough understanding of the invention. Nevertheless, the foregoing descriptions of the preferred embodiments of the present invention are presented for purposes of illustration and description and are not intended to be exhaustive or to limit the invention to the precise forms disclosed; obvious modifications and variations are possible in view of the above teachings. Accordingly, it is intended that the scope of the invention be defined by the following claims and their equivalents.

### Example 4

Air-Interface Chamber Agitation

Using the microfluidic chamber shown in FIG. 16, a food dye test was conducted, in which 1.3 μl of dye was introduced into one corner of each chamber. The total thickness of fluid in the chamber was 0.7 mm. The microfluidic chamber was mixed at 300 rpm. Complete mixing being achieved in about 10 seconds. This dye test also showed that the fluid is significantly thinner at the center during mixing because of centrifugal force on the fluid. Thus, a lower speed or pulsed shaking may be preferable.

What is claimed is:

1. A microfluidic system comprising:
(a) a microfluidic chamber comprising a flexible membrane adhered to a first surface of a substrate, and a first port; and
(b) a mixer.

2. A microfluidic system comprising:
(a) a microfluidic chamber enclosing an area of a first surface of a substrate; and
(b) a micro-disk in fluidic communication with said chamber.

3. The microfluidic system of claim 1, wherein said flexible membrane comprises a dome.

4. The microfluidic system of claim 1, wherein said flexible membrane is supported by a reinforcement structure.

5. The microfluidic system of claim 1, wherein said flexible membrane comprises polypropylene.

6. The microfluidic system of claim 1, wherein said mixer is a micro-disk in fluidic communication with said chamber.

7. The microfluidic system of claim 2 or 6, wherein said micro-disk is regulated by a magnetic field.

8. The microfluidic system of claim 7, wherein said substrate further comprises a magnetic field generator.

9. the microfluidic system of claim 7, wherein said system further comprises a magnetic field generator.

10. The microfluidic system of claim 1, wherein said mixer is positioned to apply a force to said flexible membrane.

11. The microfluidic system of claim 10, wherein said force is selected from the group consisting of centrifugal, lateral, rotational, vertical, and horizontal.

12. The microfluidic system of claim 10, wherein said force is a variable force.

13. The microfluidic system of claim 10, wherein said force is directly applied to said flexible membrane.

14. The microfluidic system of claim 10, wherein said force is indirectly applied to said flexible membrane.

15. The microfluidic system of claim 10, wherein said force distorts said flexible membrane.

16. The microfluidic system of claim 10, wherein said mixer is a shaker.

17. The microfluidic system of claim 10, wherein said mixer is a rotator.

18. The microfluidic system of claim 1 or 2, wherein said substrate comprises a material selected from the group consisting of ceramic, glass, silicon, and plastic.

19. The microfluidic system of claim 1 or 2, wherein said substrate comprises an array of capture probes.

20. The microfluidic system of claim 1 or 2, wherein said chamber further encloses an array of capture probes.

21. The microfluidic system of claim 1 or 2, wherein said chamber further comprises a second port.

22. A microfluidic system comprising:
(a) a microfluidic chamber comprising a membrane adhered to a first surface of a substrate, a spacer, and a first port; and
(b) a mixer.

23. The microfluidic system of claim 22, wherein said chamber contains a fluid and a contiguous gap between said fluid and said membrane.

24. The microfluidic system of claim 22, wherein said chamber comprises an inner surface comprising hydrophilic and hydrophobic regions.

25. The microfluidic system of claim 22, wherein said spacer comprises a shim comprising a low surface energy plastic.

26. The microfluidic system of claim 25, wherein said plastic is selected from the group consisting of polyolefin and PTFE.

27. The microfluidic system of claim 22, wherein said membrane and said spacer are contiguous.

28. The microfluidic system of claim 22, wherein said mixer is selected from the group consisting of a shaker and a rotator.

29. The microfluidic system of claim 22, wherein said mixer is a micro-disk in fluidic communication with said chamber.

30. The microfluidic system of claim 22, wherein said mixer applies a force selected from the group consisting of centrifugal, lateral, rotational, vertical, and horizontal.

31. The microfluidic system of claim 30, wherein said force is a variable force.

32. The microfluidic system of claim 22, wherein said substrate comprises a material selected from the group consisting of ceramic, glass, silicon, and plastic.

33. The microfluidic system of claim 22, wherein said substrate comprises an array of capture probes.

34. The microfluidic system of claim 22, wherein said chamber further encloses an array of capture probes.

35. The microfluidic system of claim 22, wherein said chamber further comprises a second port.

36. A microfluidic system comprising:
(a) first and second microfluidic chambers comprising a flexible membrane, and first and second substrates, wherein opposite sides of said membrane are adhered to said first and second substrates and enclose first and second areas of said substrates, wherein said first and second areas are in fluidic communication, and one of said chambers comprises a first port; and
(b) a mixer.

37. A microfluidic system comprising:
(a) first and second microfluidic chambers comprising a membrane, and first and second substrates, wherein opposite sides of said membrane are adhered to said first and second substrates and enclose first and second areas of said substrates, wherein said first and second areas are in fluidic communication, and one of said chambers comprises a first port; and
(b) a micro-disk in fluidic communication with at least one chamber.

38. The microfluidic system of claim 36, wherein said flexible membrane comprises a dome.

39. The microfluidic system of claim 36, wherein said flexible membrane is supported by a reinforcement structure.

40. The microfluidic system of claim 36, wherein said flexible membrane comprises polypropylene.

41. The microfluidic system of claim 36, wherein said mixer is a micro-disk in fluidic communication with said chamber.

42. The microfluidic system of claim 37 or 41, wherein said micro-disk is regulated by a magnetic field.

43. The microfluidic system of claim 42, wherein said substrate further comprises a magnetic field generator.

44. The microfluidic system of claim 42, wherein said system further comprises a magnetic field generator.

45. The microfluidic system of claim 36, wherein said mixer is positioned to apply a force to said flexible membrane.

46. The microfluidic system of claim 45, wherein said force is selected from the group consisting of centrifugal, lateral, rotational, vertical, and horizontal.

47. The microfluidic system of claim 45, wherein said force is a variable force.

48. The microfluidic system of claim 45, wherein said force is directly applied to said flexible membrane.

49. The microfluidic system of claim 45, wherein said force is indirectly applied to said flexible membrane.

50. The microfluidic system of claim 45, wherein said force distorts said flexible membrane.

51. The microfluidic system of claim 45, wherein said mixer is a shaker.

52. The microfluidic system of claim 45, wherein said mixer is a rotator.

53. The microfluidic system of claim 36 or 37, wherein said substrate comprises a material selected from the group consisting of ceramic, glass, silicon, and plastic.

54. The microfluidic system of claim 36 or 37, wherein said substrate comprises an array of capture probes.

55. The microfluidic system of claim 36 or 37, wherein said chamber further encloses an array of capture probes.

56. The microfluidic system of claim 36 or 37, wherein said chamber further comprises a second port.

57. A microfluidic system comprising:
(a) first and second microfluidic chambers comprising a flexible membrane, and a substrate, wherein said membrane is adhered to said substrate and encloses first and second areas of said substrate, wherein said first and second areas are in fluidic communication, and one of said chambers comprises a first port; and
(b) a mixer.

58. A microfluidic system comprising:
(a) first and second microfluidic chambers comprising a membrane, and a substrate, wherein said membrane is adhered to said substrate and encloses first and second areas of said substrate, wherein said first and second areas are in fluidic communication, and one of said chambers comprises a first port; and
(b) a micro-disk in fluidic communication with at least one chamber.

59. The microfluidic system of claim 57, wherein said flexible membrane comprises a dome.

60. The microfluidic system of claim 57, wherein said flexible membrane is supported by a reinforcement structure.

61. The microfluidic system of claim 57, wherein said flexible membrane comprises polypropylene.

62. The microfluidic system of claim 57, wherein said mixer is a micro-disk in fluidic communication with said chamber.

63. The microfluidic system of claim 58 or 62, wherein said micro-disk is regulated by a magnetic field.

64. The microfluidic system of claim 63, wherein said substrate further comprises a magnetic field generator.

65. the microfluidic system of claim 63, wherein said system further comprises a magnetic field generator.

66. The microfluidic system of claim 57, wherein said mixer is positioned to apply a force to said flexible membrane.

67. The microfluidic system of claim 66, wherein said force is selected from the group consisting of centrifugal, lateral, rotational, vertical, and horizontal.

68. The microfluidic system of claim 66, wherein said force is a variable force.

69. The microfluidic system of claim 66, wherein said force is directly applied to said flexible membrane.

70. The microfluidic system of claim 66, wherein said force is indirectly applied to said flexible membrane.

71. The microfluidic system of claim 66, wherein said force distorts said flexible membrane.

72. The microfluidic system of claim 66 wherein said mixer is a shaker.

73. The microfluidic system of claim 66, wherein said mixer is a rotator.

74. The microfluidic system of claim 57 or 58, wherein said substrate comprises a material selected from the group consisting of ceramic, glass, silicon, and plastic.

75. The microfluidic system of claim 57 or 58, wherein said substrate comprises an array of capture probes.

76. The microfluidic system of claim 57 or 58, wherein said chamber further encloses an array of capture probes.

77. The microfluidic system of claim 57 or 58, wherein said chamber further comprises a second port.

78. A method of mixing a fluid comprising:
applying a force to a flexible membrane of a microfluidic chamber containing a fluid, whereby said fluid is mixed.

79. The method of claim 78, wherein said flexible membrane is a dome.

80. The method of claim 78, wherein said flexible membrane comprises a reinforcement structure.

81. The method of claim 78, wherein said flexible membrane comprises polypropylene.

82. The method of claim 78, wherein said force is selected from the group consisting of centrifugal, lateral, rotational, vertical, and horizontal.

83. The method of claim 78, wherein said force is a variable force.

84. The method of claim 78, wherein said force is directly applied to said flexible membrane.

85. The method of claim 78, wherein said force is indirectly applied to said flexible membrane.

86. The method of claim 78, wherein said chamber further contains an array of capture probes.

87. The method of claim 78, wherein said force is applied by a mixer.

88. The method of claim 87, wherein said mixer is a shaker.

89. The method of claim 87, wherein said mixer is a rotator.

90. A method of mixing a fluid comprising:
applying a force to a fluid in a microfluidic chamber using a micro-disk in fluidic communication with said chamber, whereby said fluid is mixed.

91. The method of claim 90, wherein said chamber contains an array of capture probes.

92. The method of claim 90, wherein said chamber comprises a flexible membrane.

93. The method of claim 92, wherein said flexible membrane is a dome.

94. The method of claim 92, wherein said flexible membrane comprises a reinforcement structure.

95. The method of claim 92, wherein said flexible membrane comprises polypropylene.

96. The method of claim 90, wherein said force is a variable force.

97. A method of mixing a fluid comprising:
applying a force to a fluid in a microfluidic chamber comprising a membrane adhered to a first surface of a substrate, a spacer, a contiguous gap between said fluid and said membrane, and a first port, whereby said fluid is mixed.

98. The method of claim 97, wherein said chamber comprises an inner surface of hydrophilic and hydrophobic regions.

99. The method of claim 97, wherein said spacer comprises a low surface energy plastic.

100. The method of claim 99, wherein said plastic is selected from the group consisting of polyolefin and PTFE.

101. The method of claim 97, wherein said membrane and said spacer are contiguous.

102. The method of claim 97, wherein said force is a applied by a mixer.

103. The method of claim 102, wherein said mixer is selected from the group consisting of a shaker and rotator.

104. The method of 102, wherein said mixer applies a force selected from the group consisting of centrifugal, lateral, rotational, vertical, and horizontal.

105. The method of claim 97, wherein said force is a variable force.

106. The microfluidic system of claim 97, wherein said substrate comprises a material selected from the group consisting of ceramic, glass, silicon, and plastic.

107. The microfluidic system of claim 97, wherein said substrate comprises an array of capture probes.

108. The microfluidic system of claim 97, wherein said chamber further encloses an array of capture probes.

109. The microfluidic system of claim 97, wherein said chamber further comprises a second port.

110. A microfluidic chamber comprising a flexible membrane adhered to a first surface of a substrate, and a first port.

111. A microfluidic chamber in fluidic communication with a micro-disk.

112. A microfluidic chamber comprising a membrane adhered to a first surface of a substrate, a spacer, and a first port.

113. The microfluidic chamber of claim 112, wherein said chamber contains a fluid and a contiguous gap between said fluid and said membrane.
